(19) United States
(12) Patent Application Publication
(10) Pub. No.: US 2026/0115448 A1
(43) Pub. Date: Apr. 30, 2026

(54) ARTERIAL COMPLIANCE ADJUSTMENT

(71) Applicant: Edwards Lifesciences Corporation, Irvine, CA (US)

(72) Inventors: Roei Mayo, Irvine, CA (US); Arvin T. Chang, Yorba Linda, CA (US); Amanda Tjipta Sugijoto, Tustin, CA (US); David Lyle Leibowitz, Capistrano Beach, CA (US)

(21) Appl. No.: 19/427,369

(22) Filed: Dec. 19, 2025

Related U.S. Application Data
(63) Continuation of application No. PCT/US2024/039619, filed on Jul. 25, 2024.
(60) Provisional application No. 63/515,809, filed on Jul. 26, 2023.

(51) Int. Cl. A61M 60/295; A61M 39/00; A61M 39/22; A61M 60/135; A61M 60/30; A61M 60/497; A61M 60/843; A61M 60/859; A61M 60/892

(52) U.S. Cl. CPC A61M 60/295; A61M 39/22; A61M 60/135; A61M 60/30; A61M 60/497; A61M 60/843; A61M 60/859; A61M 60/892; A61M 2039/0009; A61M 2205/0266; A61M 2205/3331; A61M 2210/12

(57) ABSTRACT

Vascular blood flow modulation is disclosed using metallic compliance-enhancement devices that contain a sealed volume of fluid. The device includes a first elongated inflatable nitinol balloon and a second elongated inflatable nitinol balloon, each having an outer sealed terminus and an inner open fluid port, and a nitinol connector tube that fluidly couples the balloons and has a diameter smaller than the inflated diameters of the balloons. The first balloon is positioned within an arterial vessel and the second balloon is positioned within a venous vessel, with the connector tube spanning between the vessels. During systole, arterial pressure compresses the first balloon to drive fluid through the connector tube into the second balloon, thereby expanding the second balloon and increasing venous blood flow. Cyclic systolic and diastolic pressure variations cause fluid to shuttle between the balloons, producing a compliance-enhancing effect.

### CROSS REFERENCE TO RELATED APPLICATION(S)

This application is a continuation of International Patent Application No. PCT/US24/39619, filed Jul. 25, 2024, which claims the benefit of U.S. patent application Ser. No. 63/515,809, filed Jul. 26, 2023, the complete disclosures of which are hereby incorporated by reference in their entireties.

### BACKGROUND

The present disclosure generally relates to the field of medical implant devices. Insufficient or reduced compliance in certain blood vessels, including arteries such as the aorta, can result in reduced perfusion, cardiac output, and other health complications. Restoring compliance and/or otherwise controlling flow in such blood vessels can improve patient outcomes.

### SUMMARY

Described herein are methods, devices and/or systems relating to improvement of arterial compliance. In some instances, a medical compliance device can comprise a first inflatable portion and/or member configured to be positioned at a location in a venous side of the circulatory system and a second inflatable portion and/or member configured to be positioned at a location in an arterial side of the circulatory system. A medial portion extending between the first and second inflatable portions and/or members can provide fluid communication between the first and second inflatable portions and/or members to allow fluid transfer therebetween. As arterial pressure increases, fluid from the second inflatable portion and/or member can be transferred through the medial portion into the first inflatable portion and/or member, deflating the second inflatable portion and/or member and inflating the first inflatable portion and/or member. As arterial pressure decreases, fluid from the first inflatable portion and/or member can be transferred through the medial portion back into the first inflatable portion and/or member, deflating the first inflatable portion and/or member and inflating the second inflatable portion and/or member. The first inflatable portion and/or member can be in a deflated configuration while in a relaxed state, and an inflated configuration while in a tensioned state. The second inflatable portion and/or member can be in an inflated configuration while in a relaxed state, and a deflated configuration while in a tensioned state. In some instances, the first and second inflatable portions and/or members can be formed using material comprising shape-memory material, such as nitinol. Use of shape-memory material can facilitate reversion of the first and/or second inflatable portion and/or member back to their respective relaxed states, thereby providing a driving force for transferring fluid into the second inflatable portion and/or member and inflating the second inflatable portion and/or member as the arterial pressure decreases. Inflation of the second inflatable portion and/or member as arterial pressure decreases can provide improved compliance for the arterial vessel.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silico, or overlaid on one or more of the physical components. Virtual elements can be presented on any combination of screens, headsets, holographically, projected, loud speakers, headphones, pressure transducers, temperature transducers, or using any combination of suitable technologies.

For purposes of summarizing the disclosure, certain aspects, advantages and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some examples or configurations.

FIG. 1 shows an example of a medical compliance device deployed to a target site in a circulatory system, in accordance with one or more examples.

FIG. 2A shows an example of a medical compliance device comprising a first and a second inflatable balloon deployed to a target site in a circulatory system, in accordance with one or more examples.

FIGS. 2B, 2C and 2D provide a perspective view, side view and end view, respectively, of the medical compliance device described with reference to FIG. 2A.

FIG. 3A provides a perspective of a medical compliance device comprising a first and second inflatable balloon, and a valve to reduce or prevent fluid transfer between the first and second inflatable balloons during at least a portion of a diastole phase, in accordance with one or more examples. FIG. 3B shows a detailed cross-sectional view of a portion of the medical compliance device of FIG. 3A.

FIG. 4 provides a perspective view of an inflatable balloon comprising a plurality of folds configured to allow the inflatable balloon to axially expand and contract, in accordance with one or more examples.

FIG. 5 provides a perspective view of an inflatable balloon comprising a plurality of folds configured to allow the inflatable balloon to axially expand and contract, in accordance with one or more examples.

FIGS. 6A and 6B provide an end view and a side view, respectively, of an inflatable balloon comprising at least one of a first and second end portion configured to rotate around a longitudinal axis of the inflatable balloon relative to the other end portion, in accordance with one or more examples.

FIG. 7 provides a perspective view of an inflatable balloon having a ring shape, in accordance with one or more examples.

FIG. 8 is a chart showing various cross-sectional shapes of inflatable balloons that can be applicable to one or more medical compliance devices as described herein, in accordance with one or more examples.

FIGS. 9A and 9B provide side views of a medical implant system comprising a medical compliance device and a fluid reservoir, in accordance with one or more examples.

### DETAILED DESCRIPTION

The headings provided herein are for convenience only and do not necessarily affect the scope or meaning of the claimed invention.

Although certain preferred examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to the preferred examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

The vasculature of the human body, which may be referred to as the circulatory system, cardiovascular system, or vascular system, contains a complex network of blood vessels with various structures and functions and includes various veins (venous system) and arteries (arterial system). Generally, arteries, such as the aorta, carry blood away from the heart, whereas veins, such as the inferior and superior venae cavae, carry blood back to the heart. Arteries, such as the aorta, may utilize blood vessel compliance (e.g., arterial compliance) to store and release energy through the stretching of blood vessel walls. The term “compliance” is used herein according to its broad and ordinary meaning, and may refer to the ability of an arterial blood vessel or prosthetic implant device to distend, expand, stretch, or otherwise deform in a manner as to increase in volume in response to increasing intraluminal pressure, and/or the tendency of a blood vessel (e.g., artery) or prosthetic implant device, or portion thereof, to recoil toward its original dimensions as intraluminal pressure decreases. Arterial compliance facilitates perfusion of organs in the body with oxygenated blood from the heart. Generally, a healthy aorta and other major arteries in the body are at least partially elastic and compliant, such that they can act as a reservoir for blood, filling up with blood when the heart contracts during systole, such as during ventricular systole, and continuing to generate pressure and push blood to the organs of the body during diastole, such as during ventricular diastole.

The systolic phase of the cardiac cycle is associated with the pumping phase of the left ventricle, while the diastolic phase of the cardiac cycle is associated with the resting or filling phase of the left ventricle. With proper arterial compliance, an increase in volume Δv will generally occur in an artery when the pressure in the artery is increased from diastole to systole. As blood is pumped into the aorta through the aortic valve, the pressure in the aorta increases and the diameter of at least a portion thereof expands. A first portion of the blood entering the aorta during systole may pass through the artery during the systolic phase, while a second portion (e.g., approximately half of the total blood volume) may be stored in the expanded volume Δv caused by compliant stretching of the blood vessel from a non-expanded diameter d₁ to an expanded diameter d₂, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of arterial compliance may have a significant effect on perfusion and/or blood pressure in some patients. For example, arteries with relatively higher compliance may be conditioned to more easily deform than lower-compliance arteries under the same pressure conditions. Compliance (C) may be calculated using the following equation, where Δv is the change in volume (e.g., in mL) of the blood vessel, and Δp is the pulse pressure from systole to diastole (e.g., in mmHg):

$$C = \frac{Δ v}{Δ p} \qquad ( 1 )$$

In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient arterial compliance, a significant risk presented in such patients is a reduction in blood supply to the heart muscle itself. For example, during systole, generally little or no blood may flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on aortic/arterial compliance.

Insufficient perfusion of the heart muscle can lead to and/or be associated with heart failure. Heart failure is a clinical syndrome characterized by certain symptoms, including breathlessness, ankle swelling, fatigue, and others. Heart failure may be accompanied by certain signs, including elevated jugular venous pressure, pulmonary crackles and peripheral edema, for example, which may be caused by structural and/or functional cardiac abnormality. Such conditions can result in reduced cardiac output and/or elevated intra-cardiac pressures at rest or during stress.

Described herein are devices, systems and methods relating to providing improved arterial compliance. In some instances, a medical compliance device can comprise a first inflatable portion and/or member configured to be positioned in a venous portion of the circulatory system and a second inflatable portion and/or member configured to be positioned in an arterial portion of the circulatory system. A medial portion can extend between the first and second inflatable portions and/or members to provide fluid communication between the first and second inflatable portions and/or members. As arterial pressure increases, such as during systole, fluid from the second inflatable portion and/or member can be transferred through the medial portion into the first inflatable portion and/or member, deflating the second inflatable portion and/or member and inflating the first inflatable portion and/or member. As arterial pressure decreases, such as during diastole, fluid from the first inflatable portion and/or member can be transferred through the medial portion back into the second inflatable portion and/or member, deflating the first inflatable portion and/or member and inflating the second inflatable portion and/or member. Transfer of fluid between the first and second inflatable portions and/or members can provide improved compliance to the circulatory system. The first inflatable portion and/or member can be in a deflated configuration while in a relaxed state, and an inflated configuration while in a tensioned state. The second inflatable portion and/or member can be in an inflated configuration while in a relaxed state, and a deflated configuration while in a tensioned state. In some instances, the first inflatable portion and/or member can comprise a first inflatable balloon. The first inflatable balloon can comprise a first internal volume configured to receive and/or retain at least a portion of the fluid. The second inflatable portion and/or member can comprise a second inflatable balloon. The second inflatable balloon can comprise a second internal volume configured to receive and/or retain at least a portion of the fluid. The medial portion can comprise a tubular portion defining a conduit and/or lumen to allow fluid flow between the first and second inflatable balloons. In some instances, the first and second inflatable portions and/or members can be formed using material comprising shape-memory material, such as nitinol. Use of shape-memory material can facilitate reversion of the first and/or second inflatable portion and/or member back to their respective relaxed states, thereby providing a driving force for transfer of fluid between the first and second inflatable portions and/or members. For example, reversion back to the relaxed state can facilitate inflation of the second inflatable portion and/or member as the arterial pressure decreases. Inflation of the second inflatable portion and/or member as arterial pressure decreases can provide improved compliance for the arterial vessel.

In some instances, the first inflatable portion and/or member can be configured to be disposed within a portion of an inferior vena cava and the second inflatable portion and/or member can be configured to be disposed within a portion of an aorta proximate and/or adjacent to the portion of the inferior vena cava. As described in further detail herein, in some instances, the medical compliance device can be deployed using a transcaval approach. Alternatively, the first and second inflatable portions and/or members can be positioned in other proximate and/or adjacent vessels/lumens/chambers that have different pressures. For example, the first and second inflatable portions and/or members can be disposed in a portion of a superior vena cava that is proximate and/or adjacent to a portion of a right pulmonary artery, and/or a portion of a left atrium that is proximate and/or adjacent to a portion of a right atrium.

Although the medical compliance devices and/or systems described herein are described as being inserted into the femoral vein for delivery to a target site, it will be understood that the medical compliance devices and/or systems can be inserted into any number of other vessels and/or lumens. In some alternative instances, the medical compliance devices can be inserted into any number of other vessels and/or lumens for minimally invasive transcatheter delivery to another target location and/or via other delivery pathways. It will be understood that one or more components of the medical compliance devices and/or systems can undergo various processes in preparation for their use in the procedures, including for example sterilization processes. The medical compliance devices and/or systems can be sterilized medical compliance devices and/or systems.

As used herein, a “delivery lumen” can refer to any number of lumens, channels, passages, and/or connectors through which a medical instrument and/or medical device can be advanced, including for example a working channel. For example, a “delivery lumen” as used herein can refer to a working channel through which a medical instrument and/or medical device can be advanced for positioning the medical instrument and/or medical device at a target site within a patient.

Where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silico, or overlaid on one or more of the physical components. Virtual elements can be presented on any combination of screens, headsets, holographically, projected, loud speakers, headphones, pressure transducers, temperature transducers, or using any combination of suitable technologies.

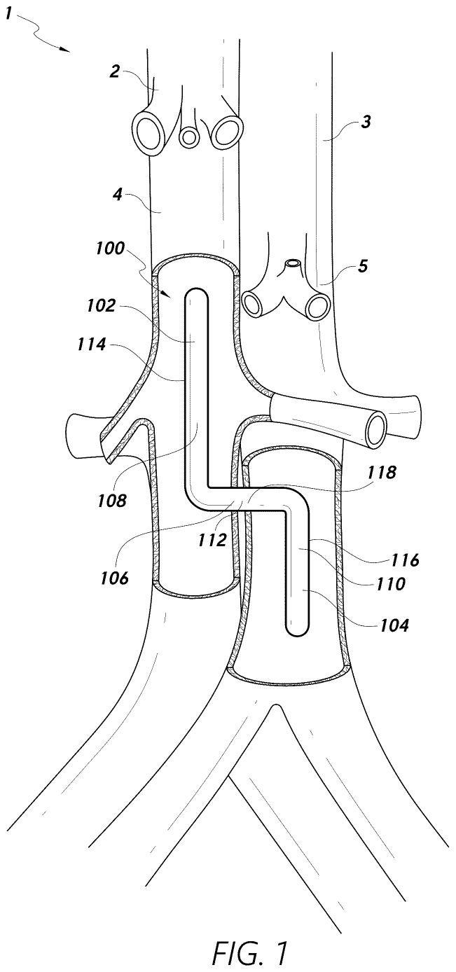

FIG. 1 shows an example of a medical compliance device 100 deployed to a target site in a circulatory system 1. The medical compliance device 100 can comprise a first inflatable portion and/or member 102 configured to be positioned in a venous portion 2 of the circulatory system 1 and a second inflatable portion and/or member 104 configured to be positioned in an arterial portion 3 of the circulatory system 1. For example, the first inflatable portion and/or member 102 can be positioned in a venous vessel, lumen and/or chamber. The second inflatable portion and/or member 104 can be positioned in an arterial vessel, lumen and/or chamber. In some instances, the medical compliance device 100 can comprise a medial portion 106 extending between the first and second inflatable portions and/or members 102, 104 to provide fluid communication between the first and second inflatable portions and/or members 102, 104. For example, the medial portion 106 can extend from the first inflatable portion and/or member 102 to the second inflatable portion and/or member 104. The medical compliance device 100 can comprise a fluid within the first inflatable portion and/or member 102, second inflatable portion and/or member 104 and/or the medial portion 106. The first inflatable portion and/or member 102 can comprise a first internal volume and/or chamber configured to receive and/or retain at least a portion of a fluid. The second inflatable portion and/or member 104 can comprise a second internal volume and/or chamber configured to receive and/or retain at least a portion of the fluid. The medial portion 106 can comprise a lumen and/or conduit to allow flow of the fluid between the first and second inflatable portions and/or members 102, 104. Transfer of fluid between the first and second inflatable portions and/or members 102, 104 can provide improved compliance for the circulatory system 1. Referring to FIG. 1, in some instances, the first inflatable portion and/or member 102 can be configured to be disposed within a portion of an inferior vena cava 4 and the second inflatable portion and/or member 104 can be configured to be disposed within a portion of an aorta 5. The medial portion 106 can comprise respective portions extending between and/or within the inferior vena cava 4 and the aorta 5. The portion of the inferior vena cava 4 and the portion of the aorta 5 can be proximate and/or adjacent to one another. In some instances, the first inflatable portion and/or member 102 can be disposed within an abdominal portion of the inferior vena cava 4. In some instances, the second inflatable portion and/or member 104 can be disposed within an abdominal portion of the aorta 5. The first inflatable portion and/or member 102 can be in a deflated configuration while in a relaxed state, and an inflated configuration while in a tensioned state. The second inflatable portion and/or member 104 can be in an inflated configuration while in a relaxed state, and a deflated configuration while in a tensioned state. As arterial pressure increases, such as during systole, fluid from the second inflatable portion and/or member 104 can be transferred through the medial portion 106 into the first inflatable portion and/or member 102, deflating the second inflatable portion and/or member 104 and inflating the first inflatable portion and/or member 102. For example, increased arterial pressure during ventricular systole can result in increased compression forces exerted upon the second inflatable portion and/or member 104. The increase in arterial pressure can compress the second inflatable portion and/or member 104, pushing fluid out of the second inflatable portion and/or member 104. As arterial pressure decreases, such as during diastole, fluid from the first inflatable portion and/or member 102 can be transferred through the medial portion 106 back into the second inflatable portion and/or member 104, deflating the first inflatable portion and/or member 102 and inflating the second inflatable portion and/or member 104. Decreased arterial pressure during ventricular diastole can result in decreased compression forces exerted upon the second inflatable portion and/or member 104. Decrease in arterial pressure can allow the second inflatable portion and/or member 104 to reinflated, returning to its relaxed state, and the first inflatable portion and/or member 102 to deflate and return to its relaxed state. Inflation of the second inflatable portion and/or member 104 as arterial pressure decreases can provide improved compliance for the arterial vessel, such as the aorta 5.

In some instances, the first and second inflatable portions and/or members can be formed using material comprising shape-memory material, such as nitinol (e.g., nitinol manufactured using physical vapor deposition (PVD) and/or molding techniques). For example, at least a portion of a wall portion 114 forming the first inflatable portion and/or member 102 and/or at least a portion of a wall portion 116 forming the second inflatable portion and/or member 104 can comprise a shape-memory material. The wall portions 114, 116 can at least partially define the first and second internal volumes and/or chambers, respectively. Compression and relaxation of the wall portions 114, 116 can cause a reduction and an increase in the first and second internal volumes and/or chambers, respectively. In some instances, the inflatable portions and/or members 102, 104 can be formed entirely or substantially entirely using shape-memory material. Use of shape-memory material can facilitate reversion of the first and/or second inflatable portion and/or member 102, 104 back to their respective relaxed states, thereby providing a driving force to deflate the first inflatable portion and/or member 102 and inflate the second inflatable portion and/or member 104 as the arterial pressure decreases. A composition of the material, thickness of the wall portions 114, 116 forming at least a portion of each inflatable portion and/or member 102, 104, and/or geometry and/or shape of the inflatable portions and/or members 102, 104 can be selected to provide the desired driving force for reverting the inflatable portions and/or members 102, 104 back to their respective relaxed states and thereby provide desired improvement in arterial compliance. In some instances, a composition of the material, thickness of the wall portions 114, 116 forming at least a portion of each inflatable portion and/or member 102, 104, and/or geometry and/or shape of the inflatable portions and/or members 102, 104 can be selected to allow desired tensioning of the inflatable portions and/or members 102, 104 under the arterial pressures. In some instances, the medial portion 106 can be formed at least in part of shape-memory material described herein. For example, a wall portion 118 at least partially forming the medial portion 106 can comprise the shape-memory material. The wall portion 118 can at least partially define the lumen and/or conduit extending between the first and second inflatable portions and/or members 102, 104. In some instances, the medical compliance device 100 can be formed entirely or substantially entirely of shape-memory material described herein.

In some instances, the medical compliance device 100 can be deployed using a transcaval approach. For example, minimally invasive transcatheter delivery of the medical compliance device 100 can comprise entry through an insertion site on a femoral vein. The medical compliance device 100 can be subsequently advanced into the inferior vena cava 4. A portion of the medical compliance device 100 can then be positioned into a portion of the aorta 5 from the inferior vena cava 4, including a portion of an abdominal aorta. For example, a portion of the medical compliance device 100 can be advanced through a wall portion of the inferior vena cava 4 and a wall portion of the aorta 5 such that the second inflatable portion and/or member 104 can be positioned into the aorta 5. In some instances, the medial portion 106 can comprise respective portions disposed through the wall portions of the inferior vena cava 4 and the aorta 5.

In some instances, the medical compliance device 100 can have a dual-balloon configuration. For example, the first inflatable portion and/or member 102 can comprise a first inflatable balloon 108. The second inflatable portion and/or member 104 can comprise a second inflatable balloon 110. The medial portion 106 can comprise a connector 112 configured to provide the fluid communication between the first and second inflatable balloons 108, 110. The connector 112 can provide a lumen and/or conduit to allow fluid flow between the first and second inflatable balloons 108, 110, including between the first and second internal volumes and/or chambers. A size and/or geometry and/or shape of the connector 112 may or may not be different from that of the first and/or second inflatable balloons 108, 110. In some instances, a lateral cross-sectional size and/or shape of the connector 112 can be smaller and/or different from that of the first and/or second inflatable balloon 108, 110. In some alternative instances, a lateral cross-sectional size and/or shape of the connector 112 may or may not be different from that of the first and/or second inflatable balloon 108, 110. In some instances, a lateral dimension of the connector 112 can be smaller than a lateral dimension of at least one of the first and second inflatable balloons 108, 110. In some instances, a lateral dimension of the connector 112 can be the same as or similar to a lateral dimension of at least one of the first and second inflatable balloons 108, 110. The lateral cross section and/or dimension can be taken along a direction perpendicular or substantially perpendicular to an axis extending between the first and second inflatable balloons 108, 110.

The first and second inflatable portions and/or members 102, 104 can have an orientation in the respective vessel so as to not occlude the vessel. In some instances, the first and second inflatable portions and/or members 102, 104 can have an orientation aligned with a direction of the blood flow through the respective vessel, for example being parallel or substantially parallel with the direction of the blood flow. Although FIG. 1 shows the first and second inflatable portions and/or members 102, 104 having opposing orientations while deployed in the circulatory system 1, it will be understood that other orientations can be applicable. For example, the first and second inflatable portions and/or members 102, 104 can have the same or similar orientation within the inferior vena cava 4 and aorta 5, respectively.

In alternative instances, the medical compliance device 100 can comprise respective portions positioned in other proximate and/or adjacent vessels/lumens/chambers having different pressures. For example, the first inflatable portion and/or member 102 of the medical compliance device 100 can be configured to be disposed within a portion of a superior vena cava and the second inflatable portion and/or member 104 can be configured to be disposed in a portion of a right pulmonary artery proximate and/or adjacent to the portion of the superior vena cava. In some instances, the first inflatable portion and/or member 102 can be disposed within a portion of a left atrium and the second inflatable portion and/or member 104 can be configured to be disposed in a portion of a right atrium proximate and/or adjacent to the portion of the left atrium.

In some instances, the fluid can comprise a liquid (e.g., saline solution). In some instances, the fluid can comprise a gas. In some instances, a viscosity of a liquid can be selected to achieve a desired response in fluid flow based at least in part on the change in arterial pressure. In some instances, the fluid can be pre-loaded within the medical compliance device 100 prior to insertion of the medical compliance device 100 into an anatomical pathway. For example, the medical compliance device 100 containing a pre-loaded amount of fluid can be implanted at the target site. In some instances, the medical compliance device 100 can be pre-filled with the fluid prior to implantation at the target site, including prior to insertion of the medical compliance device 100 into the vasculature. In some instances, the medical compliance device 100 can be sealed with the fluid contained therein prior to implantation at the target site, including prior to insertion into the vasculature. For example, the medical compliance device 100 pre-loaded with the fluid can provide the desired improvement in arterial compliance for the vasculature.

Alternatively, the fluid can be injected into the medical compliance device 100 during and/or after implantation at the target location. Advancement of the medical compliance device 100 through the anatomical pathway without or with a reduced amount of fluid preloaded therein can provide a device having a reduced profile to facilitate delivery. In some instances, injection of fluid into the medical compliance device 100 while within the vasculature, such as during or post implantation, such as after positioning at the target site, can facilitate use of an amount of fluid adjusted based at least in part on a need of a particular patient, providing patient-adjusted therapy. A valve, including a shut-off valve, can be integrated into the medical compliance device 100 to facilitate delivery of a desired amount of fluid.

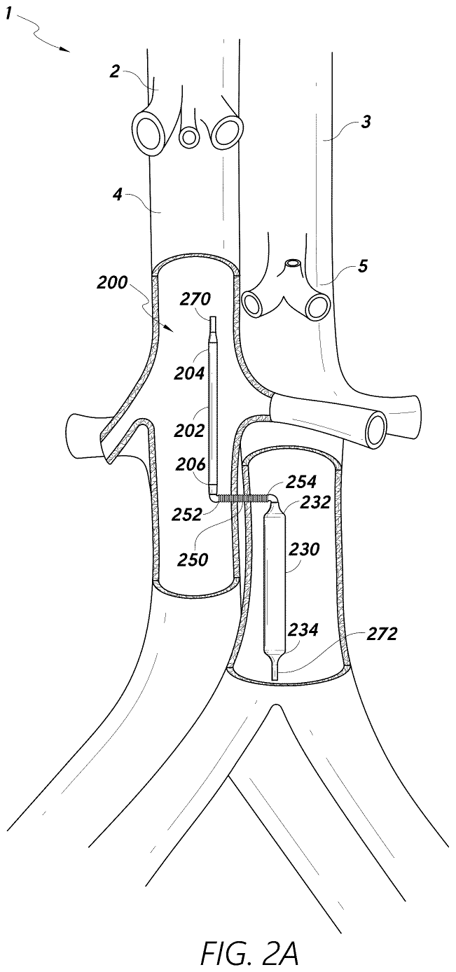

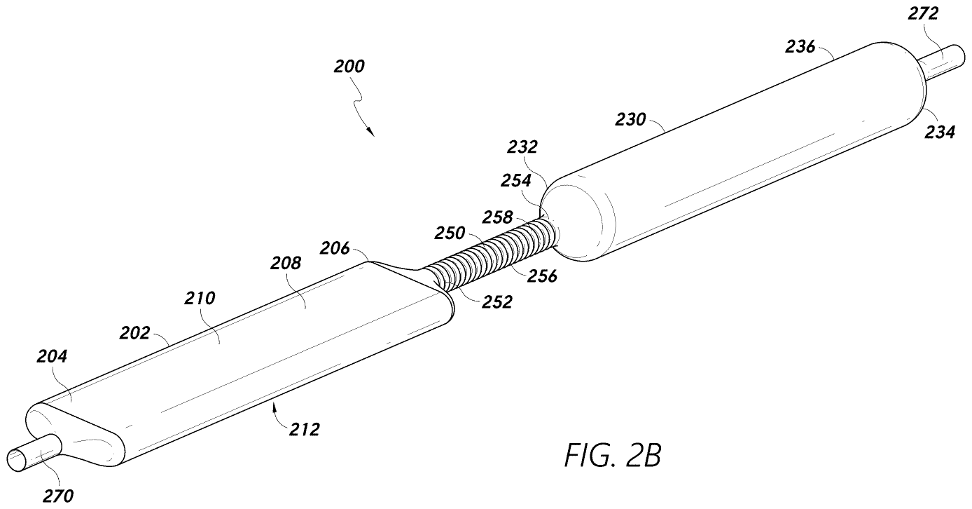

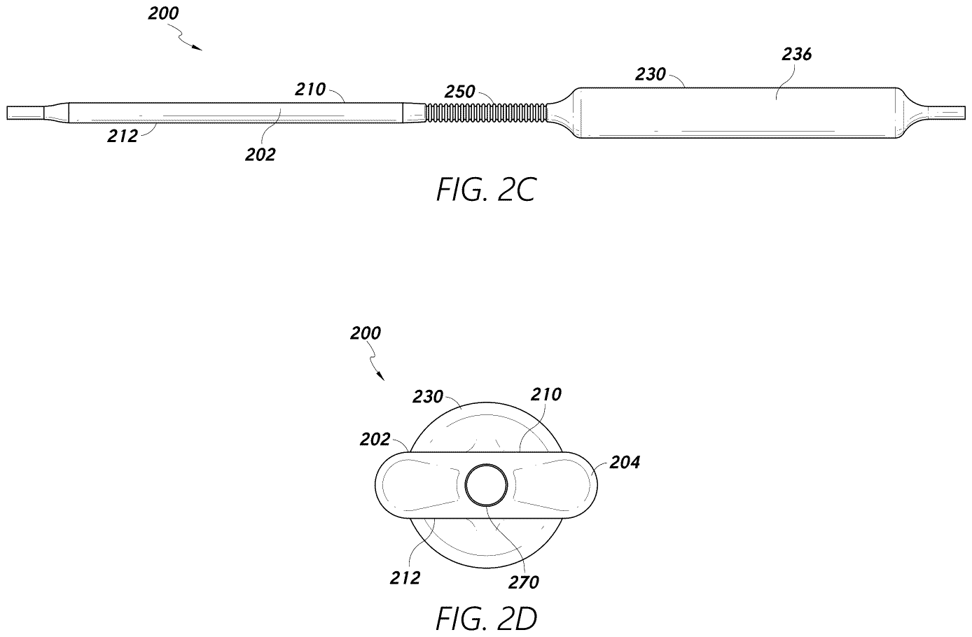

FIGS. 2A through 2D show various views of an example of a medical compliance device 200. In some instances, the medical compliance device 200 can be an example of the medical compliance device 100 described with reference to FIG. 1. For example, a first inflatable portion and/or member of the medical compliance device 200 can comprise a first inflatable balloon 202. A second inflatable portion and/or member of the medical compliance device 200 can comprise a second inflatable balloon 230. FIG. 2A shows the medical compliance device 200 disposed at a target site in the circulatory system 1. FIG. 2B shows a perspective view, FIG. 2C shows a side view, and FIG. 2D shows an end view, of the medical compliance device 200. Referring to FIG. 2A, the medical compliance device 200 can comprise the first inflatable balloon 202, the second inflatable balloon 230 and a connector 250 extending between the first and second inflatable balloons 202, 230. In some instances, the first inflatable balloon 202 can be configured to be deployed to a position in a venous side 2 of a circulatory system 1. The second inflatable balloon 230 can be configured to be deployed to a position in an arterial side 3 of the circulatory system 1. In some instances, the first inflatable balloon 202 can be configured to be disposed within a portion of an inferior vena cava 4 and the second inflatable balloon 230 can be configured to be disposed within a portion of an aorta 5. The connector 250 can comprise respective portions extending between and/or within the inferior vena cava 4 and the aorta 5. The portion of the inferior vena cava 4 and the portion of the aorta 5 can be proximate and/or adjacent to one another. In some instances, the first inflatable balloon 202 can be disposed within an abdominal portion of the inferior vena cava 4, including a portion that is adjacent and/or at a renal vein. In some instances, the second inflatable balloon 230 can be disposed within an abdominal portion of the aorta 5, including a portion that is adjacent and/or at a renal artery.

The first inflatable balloon 202 can be completely disposed, received, accommodated and/or contained within the arterial vessel, lumen and/or chamber. The second inflatable balloon 230 can be completely disposed, received, accommodated and/or contained within the venous vessel, lumen and/or chamber. For example, the first inflatable balloon 202 can be completely disposed, received, accommodated and/or contained within a respective abdominal portion of the inferior vena cava 4. The second inflatable balloon 230 can be completely disposed, received, accommodated and/or contained within a respective abdominal portion of the aorta 5, such as a portion of the abdominal aorta that is adjacent to the abdominal portion of the inferior vena cava 4. The first inflatable balloon 202 and/or second inflatable balloon 230 can be sized and/or dimensioned to provide arterial compliance without obstructing and/or hindering organ perfusion. For example, the first and/or second inflatable balloons 202, 230 can be sized and/or dimensioned to provide desired compliance while allowing sufficient blood flow around and/or past the medical compliance device 200. In some instances, a lateral dimension, including a widest lateral dimension, of the first and/or second inflatable balloon 202, 230 can be about 10 millimeters (mm) to about 20 millimeters (mm), including about 15 millimeters (mm). In some instances, a longitudinal dimension, including a longest longitudinal dimension, of the first and/or second inflatable balloon 202, 230 can be about 60 millimeters (mm) to about 120 millimeters (mm), including about 70 millimeters (mm) to about 110 millimeters (mm), about 80 millimeters (mm) to about 100 millimeters (mm), including about 90 millimeters (mm). The longitudinal dimension can be parallel or substantially parallel to a longitudinal axis of the respective balloon. The lateral dimension can be perpendicular or substantially perpendicular to the longitudinal dimension. In some instances, a lateral cross-sectional size can be less than about half that of the vessel, lumen and/or chamber.

The first and second inflatable balloons 202, 230 can have an orientation in the respective vessel so as to not occlude the vessel. In some instances, the first and second inflatable balloons 202, 230 can have an orientation aligned with that of the blood flow through the respective vessel. For example, the longitudinal axis of the first and second inflatable balloons 202, 230 can be aligned with that of the blood flow, including being parallel or substantially parallel with the direction of blood flow. While at the target site in the circulatory system 1, the connector 250 can have an orientation that is at an angle relative to that of the first and/or second inflatable balloons 202, 230. In some instances, the connector 250 can be perpendicular or substantially perpendicular relative to that of the first and/or second inflatable balloons 202, 230. For example, the longitudinal axis of the connector 250 can be at an angle, including being perpendicular or substantially perpendicular, relative to that of the first and/or second inflatable balloons 202, 230. In some instances, the first and second inflatable balloons 202, 230 can have opposing orientations, such as shown in FIG. 2A, while deployed in the circulatory system 1. For example, the first end portion 204 of the first inflatable balloon 202 and second end portion 234 of the second inflatable balloon 230 can be disposed and/or oriented away from one another. The medical compliance device 200 can form an “S” shape. In alternative instances, the first and second inflatable balloons 202, 230 can have a common or same orientation within the inferior vena cava 4 and aorta 5, respectively. For example, the medical compliance device 200 can form a “U” shape.

The connector 250 can assume a tubular form and define a lumen and/or conduit through which fluid can flow between the first and second inflatable balloons 202, 230. In some instances, a lateral dimension of the connector 250 can be smaller than a lateral dimension of at least one of the first and second inflatable balloons 202, 230. In some instances, the first inflatable balloon 202 can be in a deflated configuration while in a relaxed state. The first inflatable balloon 202 can be in an inflated configuration while in a tensioned state. The second inflatable balloon 230 can be in a deflated configuration while in a tensioned state, and in an inflated configuration while in a relaxed state. For example, a wall portion 208 forming at least a portion of the first inflatable balloon 202 can be in a relaxed state while the first inflatable balloon 202 is in a deflated configuration and a tensioned state while the first inflatable balloon 202 is in an inflated configuration. The wall portion 208 can define a first internal volume and/or chamber of the first inflatable balloon 202. A wall portion 236 forming at least a portion of the second inflatable balloon 230 can be in a relaxed state while the second inflatable balloon 230 is in an inflated configuration and a tensioned state while the second inflatable balloon 230 is in a deflated configuration. The wall portion 236 can define a second internal volume and/or chamber of the second inflatable balloon 230. The lumen and/or conduit of the connector 250 can provide fluid communication between the first and second internal volumes and/or chambers.

FIGS. 2A, 2B, 2C and 2D show the first and second inflatable balloons 202, 230 in relaxed states. The wall portion 208 forming at least a portion of the first inflatable balloon 202 can comprises a first and second opposing portions 210, 212 that are parallel or substantially parallel to one another while the first inflatable balloon 202 is in a deflated configuration, such as in a relaxed state. In some instances, a lateral cross section of the first inflatable balloon 202 can comprise a rectangular or substantially rectangular shape while the first inflatable balloon 202 is in the relaxed state. A lateral cross section of the second inflatable balloon 230 in a relaxed state can comprise one or more convex edges. In some instances, the lateral cross section of the second inflatable balloon 230 can comprise an oval shape, including a circular or substantially circular shape. In some instances, the second inflatable balloon 230 in the relaxed state can comprise a cylindrical shape. For example, the wall portion 236 forming at least a portion of the second inflatable balloon 230 can assume a cylindrical or substantially cylindrical shape while in the inflated configuration. The lateral cross sections can be taken along a plane perpendicular or substantially perpendicular to a longitudinal axis of the medical compliance device 200. In some instances, the lateral cross sections can be taken along a plane perpendicular or substantially perpendicular to a longitudinal axis of the respective inflatable balloon. The second inflatable balloon 230 can collapse to assume the tensioned state. For example, one or more portions of the wall portion 236 can deform inward to provide the second inflatable balloon 230 in deflated configuration. In some instances, a cross section of the first inflatable balloon 202 along a plane containing a longitudinal axis of the medical compliance device 200 and/or first inflatable balloon 202 can comprise a rectangular or substantially rectangular shape. For example, while in the relaxed state, the first inflatable balloon 202 can assume a rectangular prismatic or substantially a rectangular prismatic shape. The wall portion 208 can form the rectangular prismatic or substantially rectangular prismatic shape. In some instances, the first inflatable balloon 202 can bulge and/or swell to assume the tensioned state. For example, one or more portions of the wall portion 208 can stretch and/or deform outward to provide bulging and/or protruding portions of the first inflatable balloon 202 in the inflated configuration.

As pressure increases in the arterial vessel, fluid can be transferred through the connector 250 from the second inflatable balloon 230 positioned in the arterial vessel to the first inflatable balloon 202 positioned in the venous vessel. For example, as pressure increases in the aorta, such as during systole, the second inflatable balloon 230 can be compressed. Compression of the second inflatable balloon 230, such as compression of the wall portion 236 at least partially forming the second inflatable balloon 230, can result in pushing of the fluid inside the second inflatable balloon 230 through the connector 250 and into the first inflatable balloon 202. For example, as external pressure increases, such as from the increasing arterial pressure, the external pressure becomes sufficient to deform the second inflatable balloon 230, such as becoming greater than an internal pressure within the second inflatable balloon 230. The second inflatable balloon 230 can assume a deflated configuration as fluid is pushed out of the second inflatable balloon 230. The wall portion 236 of the medical compliance device 200 defining at least a portion of the second inflatable balloon 230 can be in a tensioned state while the second inflatable balloon 230 is in the deflated configuration. As fluid is transferred into the first inflatable balloon 202, the first inflatable balloon 202 can expand. For example, the first inflatable balloon 202 can assume an inflated configuration. The wall portion 208 defining at least a portion of the first inflatable balloon 202 can be in a tensioned state while the first inflatable balloon 202 is in the inflated configuration.

As pressure decreases in the arterial vessel, fluid can be transferred through the connector 250 from the first inflatable balloon 202 positioned in the venous vessel back into the second inflatable balloon 230 positioned in the arterial vessel. For example, as pressure decreases in the aorta, such as during diastole, the second inflatable balloon 230 can inflate as pressure exerted on the second inflatable balloon 230 decreases. Fluid can flow back into the second inflatable balloon 230 from the first inflatable balloon 202. The second inflatable balloon 230 can assume an inflated configuration, reverting back to a relaxed state. For example, the wall portion 236 defining at least a portion of the second inflatable balloon 230 can be in a relaxed state during at least a portion of diastole. The first inflatable balloon 202 can deflate as fluid flows out of the first inflatable balloon 202. For example, first inflatable balloon 202 can assume a deflated configuration during diastole, reverting back to a relaxed state. The wall portion 208 defining at least a portion of the first inflatable balloon 202 can be in a relaxed state during diastole.

The wall portions 208, 236 of the first and second inflatable balloons 202, 230 can comprise a shape-memory material, including for example nitinol (e.g., nitinol manufactured using physical vapor deposition (PVD) and/or using molding techniques). In some instances, the wall portion 208, 236 defining at least a portion of the first and/or second inflatable balloons 202, 230 can be made using a nitinol material, including a nitinol material formed using physical vapor deposition (PVD). For example, the shape-memory material of the first inflatable balloon 202 can stretch and/or expand as fluid flows into the first inflatable balloon 202 to allow the first inflatable balloon 202 to assume an inflated configuration. The shape-memory material can facilitate reversion of the first inflatable balloon 202 back to the deflated configuration as the fluid flows out. The shape-memory material of the second inflatable balloon 230 can deform as the second inflatable balloon 230 is compressed and fluid is pushed out to allow the second inflatable balloon 230 to assume a deflated configuration. The shape-memory material can facilitate reversion of the second inflatable balloon 230 back to the inflated configuration as pressure exerted upon the second inflatable balloon 230 decreases and the fluid returns. In some instances, a composition of the material, a thickness of the wall portions, and/or a shape and/or geometry of the first inflatable balloon 202 can be selected to provide desired driving force for the first inflatable balloon 202 to revert back to its relaxed state from a tensioned state to thereby push fluid back into the second inflatable balloon 230 and facilitate inflation of the second inflatable balloon 230 as the arterial pressure decreases. In some instances, a composition of the material, thickness of the wall portions forming at least a portion of each inflatable balloons 202, 230, and/or geometry and/or shape of the inflatable balloons 202, 230 can be selected to allow desired tensioning of the inflatable balloons 202, 230 under increasing arterial pressures.

As described herein, the connector 250 can assume a tubular form. A first end portion 252 of the connector 250 can be coupled to a second end portion 206 of the first inflatable balloon 202. A second end portion 254 of the connector 250 can be coupled to a first end portion 232 of the second inflatable balloon 230. For example, the connector 250 can extend from the second end portion 206 of the first inflatable balloon 202 to the first end portion 232 of the second inflatable balloon 230. In some instances, a wall portion 256 forming the connector 250 can comprise a plurality of ridges 258. Each of the plurality of ridges 258 can be circumferentially extending ridges. In some instances, each of the plurality of ridges 258 can be circumferentially disposed and be oriented perpendicularly or substantially perpendicularly relative to a longitudinal axis of the medical compliance device 200 and/or a longitudinal axis of the connector 250. In some instances, the ridges 258 can facilitate deformation of the connector 250 due to changes in the arterial pressure. For example, the ridges 258 can allow compression and/or stretching of the connector 250, such as to accommodate increase and/or decreases in the arterial pressure. In some instances, the ridges 258 can facilitate bending of the connector 250, such as to facilitate delivery and/or positioning of the medical compliance device 200 to a target site. For example, the ridges 258 can facilitate bending of one or more portions of the connector 250 to provide desired orientation of the first and/or second inflatable balloons 202, 230 at the target site. In some instances, the ridges 258 can facilitate desired positioning of the medical compliance device 200 at the target site, for example providing frictional contact with tissue at the target site.

In some instances, the medical compliance device 200 can comprise a first end tubular member 270 and/or a second end tubular member 272 configured to facilitate injection of fluid into the medical compliance device 200. The first end tubular member 270 can be coupled to a first end portion 204 of the first inflatable balloon 202. The second end tubular member 272 can be coupled to a second end portion 234 of the second inflatable balloon 230. The first and second end tubular members 270, 272 can each comprise a respective port for coupling to an injection device for delivering fluid into the respective inflatable balloon. In some instances, the first and/or second end tubular members 270, 272 can comprise a one-way valve housed therein to control fluid flow into the respective inflatable balloon. In some instances, the first and second end tubular members 270, 272 can be removed from the medical compliance device 200 after desired injection of fluid. For example, the first end portion 204 of the first inflatable balloon 202 can form a first end portion of the medical compliance device 200 and the second end portion 234 of the second inflatable balloon 230 can form a second end portion of the medical compliance device 200. In some instances, a pre-filled medical compliance device 200 does not comprise the first and second end tubular members 270, 272.

FIGS. 2C and 2D show a side view and an end view of the medical compliance device 200 in the relaxed state, respectively. The first and second inflatable balloons 202, 230 are shown in a deflated and inflated configuration, respectively. For example, the opposing wall portions 210, 212 of the first inflatable balloon 202 can be parallel or substantially parallel to one another. The wall portion 236 defining at least a portion of the second inflatable balloon 230 can assume a cylindrical shape. FIG. 2D shows the medical compliance device from the first end portion 204 of the first inflatable balloon 202.

In some instances, the medical compliance device 200 can be configured to provide desired improvement in arterial compliance, such as without use of any additional mechanisms to facilitate desired inflation and/or deflation of the inflatable balloons 202, 230. For example, the medical compliance device 200 can provide desired improvement in compliance without use of stents in combination with the inflatable balloons 202, 230. In some instances, the medical compliance device 200 can be sealed with the fluid contained therein prior to implantation at the target site, including prior to insertion into the vasculature. For example, deployment of the medical compliance device 200 pre-loaded with the fluid can provide the desired improvement in arterial compliance for the vasculature. In some instances, fluid can be injected into the medical compliance device 200 during and/or after implantation at the target location. For example, the medical compliance device 200 can be deployed to or proximate to the target site while empty or containing an initial amount of fluid. An amount of fluid can be added to the medical compliance device 200 while the medical compliance device 200 is in the vasculature. In some instances, one or more of the inflatable balloons 202, 230 can be used in combination with one or more stents (e.g., coupled to at least a portion of walls forming the inflatable balloons 202, 230) to facilitate deflating the first inflatable balloon 202 and/or inflating of the second inflatable balloon 230.

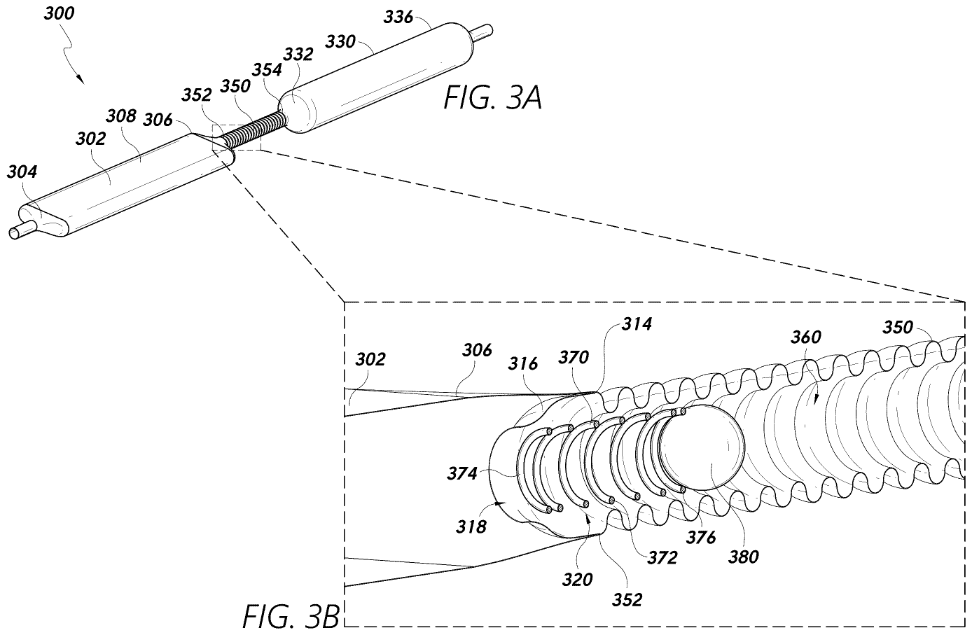

FIGS. 3A and 3B show an example of a medical compliance device 300 comprising a valve 370. FIG. 3A shows a perspective view of the medical compliance device 300 and FIG. 3B shows a detailed cross-sectional view of a portion of the medical compliance device 300. Referring to FIG. 3A, the medical compliance device 300 can comprise a first inflatable balloon 302, a second inflatable balloon 330 and a connector 350 extending between the first and second inflatable balloons 302, 330. The first inflatable balloon 302 can be configured to be deployed to a position in a venous side of a circulatory system. The second inflatable balloon 330 can be configured to be deployed to a position in an arterial side of the circulatory system. In some instances, the first inflatable balloon 302 can be in a deflated configuration while in a relaxed state and in an inflated configuration while in a tensioned state. The second inflatable balloon 330 can be in a deflated configuration while in a tensioned state, and in an inflated configuration while in a relaxed state. The connector 350 can define a conduit and/or lumen 360 configured to allow fluid flow between the first and second inflatable balloons 302, 330 to inflate and/or deflate the balloons. The connector 350 can comprise a first end portion 352 oriented toward and/or coupled to a second end portion 306 of the first inflatable balloon 302, and a second end portion 354 oriented toward and/or coupled to a first end portion 332 of the second inflatable balloon 330. The connector 350 can define a lumen 360 extending between the first and second inflatable balloons 302, 330. In some instances, the connector 350 can extend from the second end portion 306 of the first inflatable balloon 302 to the first end portion 332 of the second inflatable balloon 330.

Referring to FIG. 3B, at least a portion of the valve 370 can be received within the connector 350, for example within the conduit and/or lumen 360 of the connector 350. The valve 370 can be configured to remain closed while the arterial pressure is below a threshold value and assume an open state when the arterial pressure is at and/or above the threshold. While the valve 370 is closed, the valve 370 can be configured to reduce or prevent fluid flow into the first inflatable balloon 302. For example, the valve 370 can reduce or prevent fluid flow from the second inflatable balloon 330 into the first inflatable balloon 302 during at least a portion the diastole phase, thereby reducing or preventing loss in compliance improvement due to diastole loss. In some instances, the threshold pressure value can be about 50 millimeters of mercury (mmHg) to about 70 millimeters of mercury (mmHg), including about 60 millimeters of mercury (mmHg). For example, if the arterial pressure if less than about 60 millimeters of mercury (mmHg), the valve 370 can remain closed. If the arterial pressure is at or above about 60 millimeters of mercury (mmHg), the valve 370 can open. In some instances, flow through the valve 370 can depend on an arterial pressure, such as increasing with increasing arterial pressure. In some instances, flow through the valve 370 can be proportional to arterial pressure, for example being directly related to the arterial pressure. In some instances, the valve 370 can be in an open state during systole such that fluid can be transferred from the second inflatable balloon 330 into the first inflatable balloon 302. In some instances, the valve 370 can assume a closed state during at least a portion of the diastole phase to reduce or prevent transfer of fluid into the first inflatable balloon 302.

In some instances, the valve 370 can comprise a spring 372, such as a coil spring, and an occluding member, such as a ball 380. The spring 372 can have an orientation parallel or substantially parallel to a longitudinal axis of the medical compliance device 300 and/or connector 350. For example, a first end 374 of the spring 372 can be oriented toward a first end portion 304 of the first inflatable balloon 302. A second end 376 of the spring 372 can be oriented toward the second inflatable balloon 330, such as being opposingly oriented relative to the first end 374. The ball 380 can be disposed over and/or against the second end 376 of the spring 372. The valve 370 can be closed in a relaxed state. For example, while the spring 372 is in a relaxed state, the ball 380 can be disposed at a position along the fluid flow pathway between the first and second inflatable balloons 302, 330 so as to occlude fluid flow therebetween. While in a tensioned state, the valve 370 can be open to allow fluid flow therethrough. For example, as the spring 372 is compressed, such as when arterial pressure increases and pressure is applied upon the second inflatable balloon 330 and/or the connector 350, the ball 380 can be translated along with the second end 376 of the spring 372 toward the first end 374 of the spring 372 until the ball 380 is disposed at a position along the fluid flow pathway such that the ball 380 no longer occludes fluid flow. While the valve 370 is in an open state, the ball 380 can be positioned at a location within the lumen 360 and/or a lumen and/or chamber defined at least in part by the first inflatable balloon 302 that has a diameter larger than that of the ball 380.

Referring again to FIG. 3B, a wall portion 316 forming at least a portion of the second end portion 306 of the first inflatable balloon 302 can taper inward toward a longitudinal axis of the medical compliance device 300 and/or longitudinal axis of the first inflatable balloon 302. The wall portion 316 can extend from a second end 314 of the first inflatable balloon 302 toward the first end portion 304 of the first inflatable balloon 302, while tapering inward toward the longitudinal axis. For example, the wall portion 316 can form a funnel-shaped lumen portion 320. An end of the wall portion 316 oriented toward the first end portion 304, and away from the second end 314, can define an opening 318. The opening 318 can be at a smaller of two ends of the funnel-shaped lumen portion 320, and can be configured to allow fluid flow therethrough into an internal volume of the first inflatable balloon 302. A portion of the funnel-shaped lumen portion 320 oriented toward the connector 350 can have a lateral dimension, such as diameter, larger than that of the conduit and/or lumen 360. In some instances, while the valve 370 is in a relaxed state, a portion of the valve 370 can be housed within a portion of the conduit and/or lumen 360 at the first end portion 352 of the connector 350. Another portion of the valve 370 can be received within the funnel-shaped lumen portion 320. While in the relaxed state, a portion of the spring 372 can be received within the funnel-shaped lumen portion 320 and another portion of the spring 372 can be in the conduit and/or lumen 360. For example, the first end 374 of the spring 372 can be within the funnel-shaped lumen portion 320 and the second end 376 can be in the conduit and/or lumen 360. The first end 374 can be positioned over and/or against the wall portion 316. While in the relaxed state, the ball 380 can be within the conduit and/or lumen 360 to reduce or prevent flow therethrough. For example, the ball 380 can be within the conduit and/or lumen 360 to occlude the conduit and/or lumen 360, providing the valve 370 in the closed state. As the arterial pressure increases, the spring 372 can compressed such that the second end 376 is moved toward the first end 374. Compression of the spring 372 can cause the ball 380 sitting against the second end 376 to be pushed into the funnel-shaped lumen portion 320, thereby removing the occlusion in the conduit and/or lumen 360 and allowing fluid to flow through the conduit and/or lumen 360 and through the opening 318 into the first inflatable balloon 302. The valve 370 can thereby assume an open state. In some instances, the ball 380 can be at least partially disposed within the funnel-shaped portion 320 at or above a threshold arterial pressure, including for example, about 50 millimeters of mercury (mmHg) to about 70 millimeters of mercury (mmHg), including about 60 millimeters of mercury (mmHg). Positioning the ball 380 into a portion of the fluid flow pathway having a lateral dimension, such as diameter, larger than that of the ball 380 can facilitate transitioning the valve 370 into an open state and such that the ball 380 does not occlude the fluid flow pathway. As arterial pressure decreases, the spring 372 can return to its relaxed state, pushing the ball 380 back into the conduit and/or lumen 360.

In some instances, one or more portions of the spring 372 can engage with and/or be coupled to one or more respective portions of the connector 350. In some instances, an increase in arterial pressure can provide forces to compress the medical compliance device 300, including the connector 350. In some instances, forces exerted by the increasing arterial pressure upon the connector 350 and/or the medical compliance device 300 can shorten the connector 350. Compression, such as shortening, of the connector 350 along its longitudinal dimension can result in compression of the spring 372 disposed within the connector 350. For example, shortening of the connector 350 can result in exertion of compression forces to upon the spring 372, such as to move the second end 376 toward the first end 374, and/or vice versa. Shortening of the spring 372 along its longitudinal dimension can continue as the arterial pressure increases, such as until the ball 380 is positioned within the funnel-shaped lumen portion 320. In some instances, as the arterial pressure decreases, compression forces exerted upon the connector 350 and/or medical compliance device 300 can decrease, thereby decreasing compression forces exerted upon the spring 372. The spring 372 can lengthen along its longitudinal dimension, positioning the ball 380 back into the conduit and/or lumen 360 and occluding the conduit and/or lumen 360.

Providing the valve 370 to reduce or prevent fluid flow during at least a portion the diastole phase can thereby reduce or prevent volume change in the second inflatable balloon 330 during diastole, increasing volume change in the second inflatable balloon 330 during systole and reducing loss in compliance improvement due to diastole loss. The medical compliance device 300 can comprise one or more other features of the medical compliance devices 100, 200 described with reference to FIGS. 1, 2A, 2B, 2C and 2D. For example, a lateral cross section of the first inflatable balloon 302 can comprise a rectangular or substantially rectangular shape while the first inflatable balloon 302 is in the relaxed state. A lateral cross section of the second inflatable balloon 330 in a relaxed state can comprise one or more convex edges. In some instances, the lateral cross section of the second inflatable balloon 330 can comprise an oval shape, including a circular or substantially circular shape. The lateral cross sections can be taken along a plane perpendicular or substantially perpendicular to a longitudinal axis of the medical compliance device 300 and/or respective inflatable balloon 302, 330. In some instances, a cross section of the first inflatable balloon 302 along a plane containing a longitudinal axis of the first inflatable balloon 302 can comprise a rectangular or substantially rectangular shape. A wall portion 308 forming at least a portion of the first inflatable balloon 302 can assume a rectangular prismatic shape or substantially a rectangular prismatic shape while in the relaxed state. In some instances, a wall portion 336 forming at least a portion of the second inflatable balloon 330 can assume a cylindrical shape while in the relaxed state.

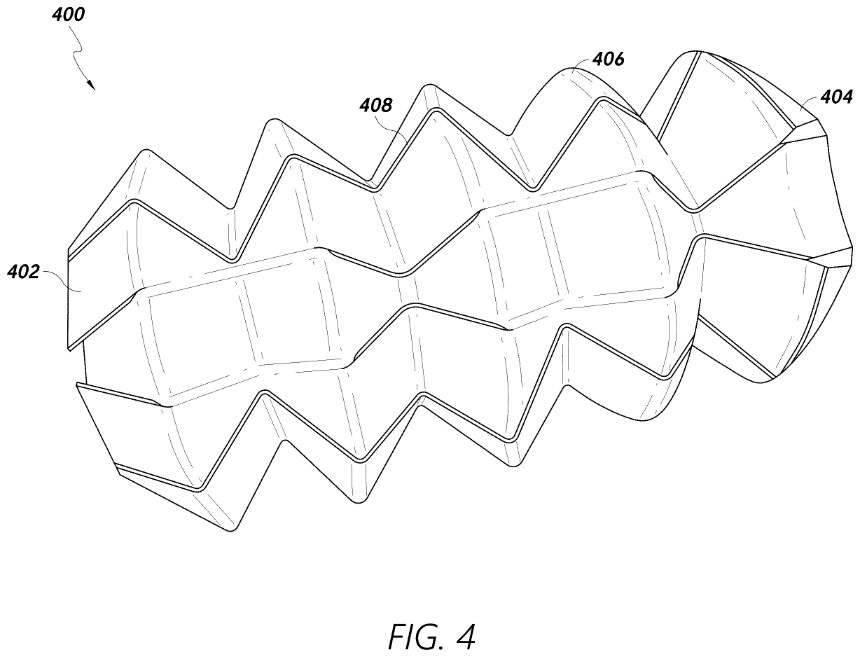

FIGS. 4, 5, 6A, 6B, 7 and 8 show various examples of inflatable balloons that can be a part of a medical compliance device. The medical compliance device can have one or more features of one or more of the medical compliance devices 100, 200, 300 described with reference to FIGS. 1, 2A, 2B, 2C, 2D, 3A and 3B. For example, the medical compliance device can comprise a first inflatable balloon, a second inflatable balloon and a connector between the first and second inflatable balloons such that the first and second inflatable balloons are in fluid communication. The first inflatable balloon can be configured to be deployed to a position on a venous side of the circulatory system, for example comprising a deflated configuration in a relaxed state. The second inflatable balloon can be configured to be deployed to a position on an arterial side of the circulatory system, for example comprising an inflated configuration in a relaxed state. In some instances, one or more of the inflatable balloons described with reference to FIGS. 4, 5, 6A, 6B, 7 and 8 can be the first inflatable balloon of the medical compliance device. For example, a medical compliance device can comprise an inflatable balloon described with reference to FIG. 4, 5, 6A, 6B, 7 or 8. A relaxed state of the inflatable balloon described with reference to FIGS. 4, 5, 6A, 6B, 7 and 8 can be a deflated configuration. One or more other features of the medical compliance device can comprise one or more features of the medical compliance devices 100, 200, 300 described with reference to FIGS. 1, 2A, 2B, 2C, 2D, 3A and 3B. Alternatively, a medical compliance device can comprise an inflatable balloon described with reference to FIG. 4, 5, 6A, 6B, 7 or 8 as the second inflatable balloon. For example, a relaxed state of the inflatable balloon described with reference to FIGS. 4, 5, 6A, 6B, 7 and 8 can be an inflated configuration. One or more other features of the medical compliance device can comprise one or more features of the medical compliance devices 100, 200, 300 described with reference to FIGS. 1, 2A, 2B, 2C, 2D, 3A and 3B.

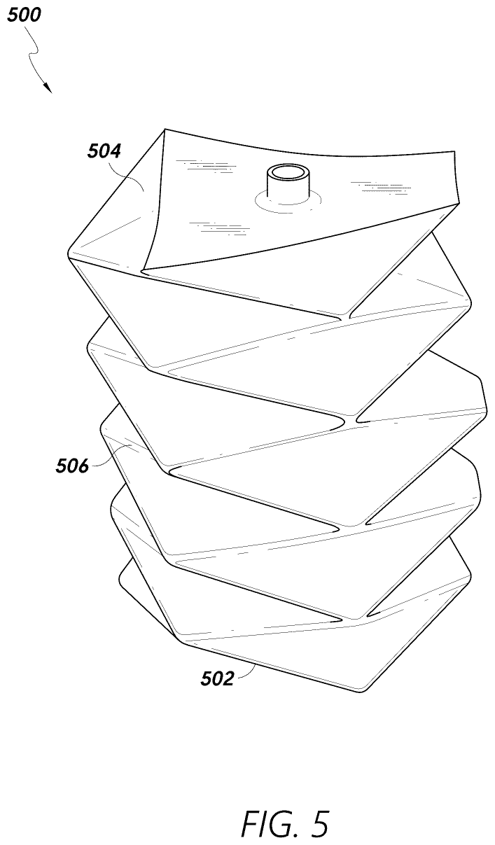

FIGS. 4 and 5 are perspective views of examples of inflatable balloons 400, 500 which can axially lengthen or shorten to increase or decrease an internal volume of the inflatable balloon 400, 500, respectively. The inflatable balloons 400, 500 can each comprise one or more folds 406, 506, including circumferentially disposed and/or oriented folds, to allow the inflatable balloons 400, 500 to expand or contract along a longitudinal axis of the respective inflatable balloon. Unfolding and folding of the plurality of folds 406, 506 can allow a first end 402, 502 to move away from or toward a second end 404, 504, respectively. The first end 402, 502 or the second end 404, 504 can be coupled to a connector of the medical compliance device. For example, the plurality of folds 406, 506 can allow the inflatable balloons 400, 500 to expand and/or contract along a linear dimension parallel or substantially parallel to the longitudinal axis of the respective inflatable balloon 400, 500. In some instances, a lateral dimension perpendicular or substantially perpendicular to the longitudinal axis, including a width and/or diameter, can remain the same or substantially the same as a longitudinal dimension along the longitudinal axis, such as a length, increases or decreases. Alternatively, the lateral dimension can decrease or increase as the length increases or decreases, respectively. The plurality of circumferentially disposed and/or oriented folds 406, 506 can have an orientation angled, including perpendicular or substantially perpendicular, relative to the longitudinal axis of the inflatable balloon 400, 500 such that unfolding and folding of the folds 406, 506 can lengthen and shorten the inflatable balloon 400, 500, respectively. The plurality of circumferentially disposed and/or oriented folds 406, 506 can form alternating concave and convex exterior angles along a longitudinal dimension of the inflatable balloons 400, 500. The inflatable balloons 400, 500 can comprise a bellow and/or accordion configuration configured to provide axial spring storing energy capabilities. FIGS. 4 and 5 show the inflatable balloons 400, 500 in deflated configurations. For example, a relaxed state of the inflatable balloons 400, 500 can comprise the deflated configuration such that the plurality of folds 406, 506 are in a folded configuration. In some instances, the inflatable balloons 400, 500 can be configured to be disposed at a location within a venous side of the circulatory system, such as the in an inferior vena cava. As fluid is transferred into the inflatable balloon 400, 500, such as while arterial pressure increases, the plurality of folds 406, 506 can unfold to allow expansion of the respective balloon along its longitudinal axis. As arterial pressure decreases and fluid leaves the inflatable balloons 400, 500, the plurality of folds 406, 506 can revert back to a folded configuration to allow reversion of the inflatable balloons 400, 500 back to the deflated configuration. Alternatively, a relaxed state of the inflatable balloons 400, 500 can comprise an inflated configuration. For example, the inflatable balloons 400, 500 can be configured to be disposed at a location within an arterial side of the circulatory system, such as the in an aorta.

Referring to FIG. 4, each of the plurality of circumferentially disposed and/or oriented folds 406 can extend circumferentially around the inflatable balloon 400. In some instances, the plurality of circumferentially disposed and/or oriented folds 406 can form one or more convex curvatures around a circumference of the inflatable balloon 400. In some instances, the plurality of circumferentially disposed and/or oriented folds 406 can form an oval shape, including a circular or substantially circular shape. In some instances, the inflatable balloon 400 can comprise a plurality of longitudinal folds 408. The plurality of longitudinal folds 408 can be at respective positions around the circumference of the inflatable balloon 400. In some instances, the plurality of longitudinal folds 408 can unfold and fold to allow an increase and/or decrease in diameter of the inflatable balloon 400. For example, the inflatable balloon 400 can widen or narrow as the plurality of longitudinal folds 408 unfold and fold, respectively, to transform the inflatable balloon 400 between inflated and deflated configurations.

Referring to FIG. 5, a circumferentially disposed and/or oriented fold 506 can extend along a portion of a circumference of the inflatable balloon 500. In some instances, a plurality of circumferentially disposed and/or oriented fold 506 can form a circumference. In some instances, a circumferentially disposed and/or oriented fold 506 can be at an angle, including being perpendicular or substantially perpendicular, relative to an adjacent circumferentially disposed and/or oriented fold 506. For example, a plurality of circumferentially disposed and/or oriented folds 506 can form a rectangularly shaped, including a square shaped, circumference.

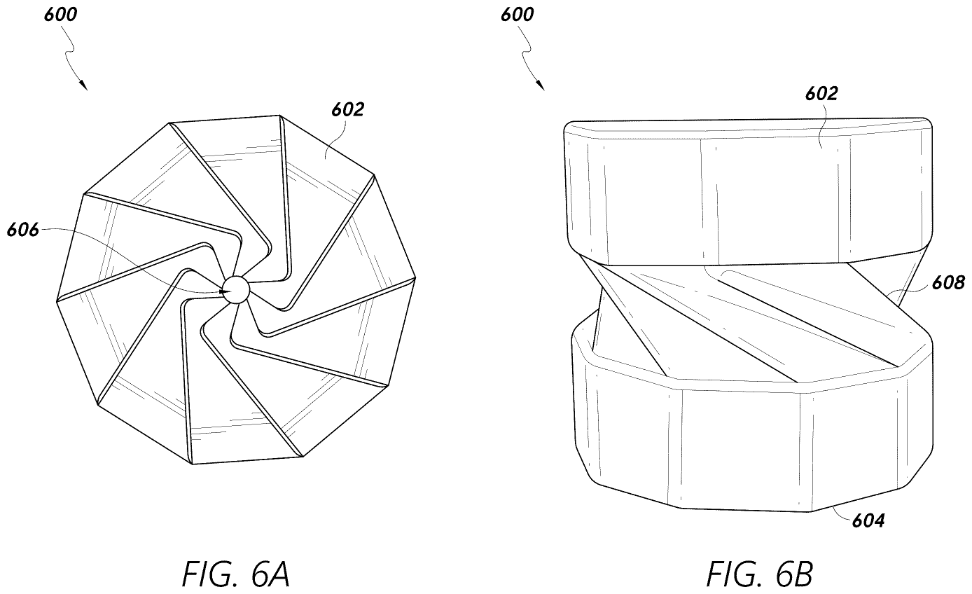

FIGS. 6A and 6B show an example of inflatable balloon 600 comprising end portions that can be rotated relative to one another around a longitudinal axis of the inflatable balloon 600 to increase or decrease an internal volume of the inflatable balloon 600. FIG. 6A shows an end view, and FIG. 6B shows a side view, of the inflatable balloon 600. Referring to FIG. 6A, a first end portion 602 of the inflatable balloon 600 is shown. An opening 606 on the first end portion 602 can be coupled to a connector to provide fluid communication between the inflatable balloon 600 and another inflatable balloon of a medical compliance device. Alternatively, the connector can be coupled to a second end portion 604 of the inflatable balloon 600. Referring to FIG. 6B, the first end portion 602 can move away from or toward the second end portion 604 of the inflatable balloon 600 as one or both of the first end portion 602 and second end portion 604 rotate around the longitudinal axis of the inflatable balloon 600. The first end portion 602 and/or the second end portion 604 can rotate around the longitudinal axis such that the two end portions 602, 604 can move away from one another, for example untwisting the inflatable balloon 600. Untwisting of the inflatable balloon 600 can result in axial lengthening of the inflatable balloon 600 and an increase in internal volume, for example lengthening along a dimension parallel or substantially parallel to the longitudinal axis of the inflatable balloon 600. In some instances, the first end portion 602 can be rotated in a first direction around the longitudinal axis while the second end portion 604 remains stationary and/or while the second portion 604 is rotated in a second direction around the longitudinal axis opposite that of the first direction so as to effect untwisting of the inflatable balloon 600. The first end portion 602 can be rotated in the second direction while the second end portion 604 is stationary and/or while the second end portion 604 is rotated in the first direction such that the two end portions 602, 604 move toward from one another, for example twisting the inflatable balloon 600. Twisting the inflatable balloon 600 can result in axial shortening of the inflatable balloon 600 and decrease in internal volume, for example shortening a length of the balloon 600 along a dimension parallel or substantially parallel to the longitudinal axis. Although FIGS. 6A and 6B show the end portions 602, 604 as comprising a rounded shape, such as a circular or substantially circular shape, it will be understood that the end portions 602, 604 can comprise any number of other shapes, including oval or polygonal.

The inflatable balloon 600 can comprise a plurality of circumferentially disposed folds 608 that unfolds or folds as the inflatable balloon 600 expands or contracts, respectively. For example, the circumferentially disposed folds 608 can unfold as the inflatable balloon 600 untwists to expand and fold as the inflatable balloon 600 twists to contract. One or more of the plurality of circumferentially disposed folds 608 can be disposed along a dimension extending between the first and second end portions 602, 604. While in the untwisted configuration, the plurality of circumferentially disposed folds 608 can extend along a dimension parallel or substantially parallel to the longitudinal axis of the inflatable balloon 600. In some instances, a relaxed state of the inflatable balloon 600 can comprise a deflated configuration such that the plurality of folds 608 are in a folded configuration. For example, the inflatable balloon 600 can be configured to be disposed at a location on a venous side of the circulatory system. As fluid is transferred into the inflatable balloon 600 the plurality of folds 608 can unfold to allow expansion of the inflatable balloon 600, such as lengthening along its longitudinal axis. As fluid leaves the inflatable balloon 600, the plurality of folds 608 can revert back to a folded configuration to allow reversion of the inflatable balloon 600 back to the deflated configuration, for example shortening along its longitudinal axis. Alternatively, the inflatable balloon 600 can assume an inflated configuration while in a relaxed state. For example, the inflatable balloon 600 can be configured to be disposed at a location on an arterial side of the circulatory system. As the inflatable balloon 600 is compressed and fluid is transferred out of the inflatable balloon 600, the plurality of folds 608 can fold to allow contraction of the inflatable balloon 600. The inflatable balloon 600 can shorten along its longitudinal axis and assume a twisted configuration. As pressure exerted upon the inflatable balloon 600 is reduced, fluid can enter the inflatable balloon 600, the plurality of folds 608 can revert back to an unfolded configuration to allow reversion of the inflatable balloon 600 back to the inflated configuration. In some instances, twisting and/or untwisting of the inflatable balloon 600 can provide axial spring storing energy capabilities.

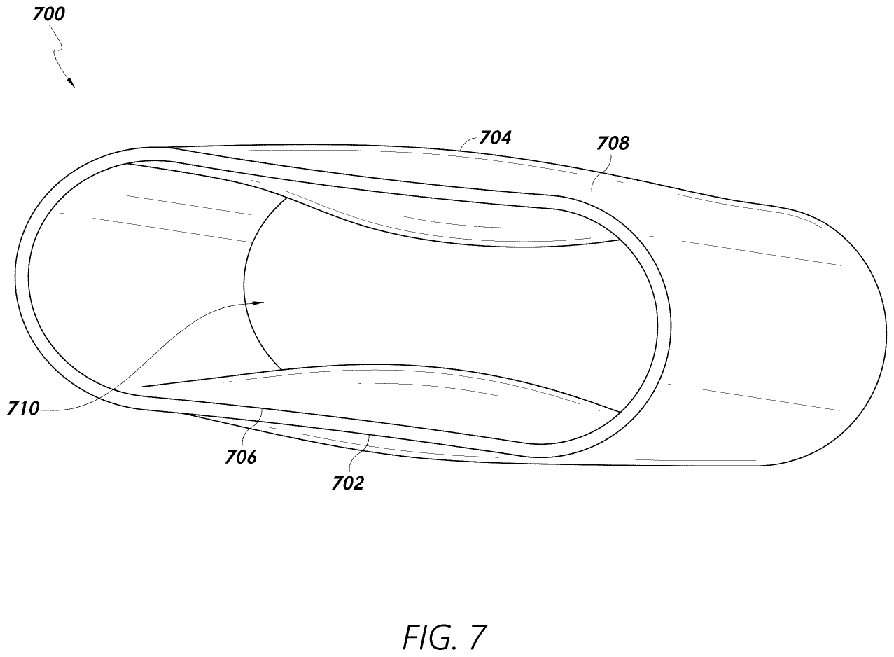

FIG. 7 shows an example of an inflatable balloon 700 having a ring shape. At least a portion of the inflatable balloon 700 can inflate and deflate. In some instances, a first ring portion 702 and a second ring portion 704 of the inflatable balloon 700 can inflate or deflate as fluid is transferred into or out of an internal volume of the inflatable balloon 700, respectively. In some instances, the first and second ring portions 702, 704 can be opposing portions, for example being at opposingly oriented positions around the circumference of the ring. In alternative instances, the inflatable balloon 700 can comprise another number of portions around the ring that can inflate and/or deflate, including three, four, five, six, etc. In some instances, the first and second ring portions 702, 704 can be at positions other than opposing positions. FIG. 7 shows the inflatable balloon 700 in an inflated configuration. In some instances, the inflatable balloon 700 can be in the inflated configuration while in a relaxed state. The first and second ring portions 702, 704 can comprise a respective portion that bulges and/or protrudes while in the inflated configuration. The inflatable balloon 700 can be configured to be disposed at a location on an arterial side of the circulatory system. For example, as the inflatable balloon 700 is compressed and fluid is transferred out of the inflatable balloon 700, opposing portions of the first wall portion 706, and opposing portions of the second wall portion 708 can move toward one another as the wall portions 706, 708 assume a tensioned state. In some instances, opposing portions of the first wall portion 706 and/or opposing portions of the second wall portion 708 can contract and/or shrink to assume the tensioned state. The inflatable balloon 700 can assume a deflated state in the tensioned state. As pressure exerted upon the inflatable balloon 700 is reduced, fluid can enter the inflatable balloon 700. Opposing portions of the first wall portion 706, and opposing portions of the second wall portion 708 can move away from one another to allow reversion of the inflatable balloon 700 back to the inflated configuration. Alternatively, in some instances, a relaxed state of the inflatable balloon 700 can comprise a deflated configuration. The inflatable balloon 700 can be configured to be disposed at a location on a venous side of the circulatory system. For example, a first wall portion 706 forming the first ring portion 702 and/or a second wall portion 708 forming the second ring portion 704 can stretch and/or expand as fluid is transferred into the inflatable balloon 700. For example, the first and/or second wall portions 706, 708 can bulge to assume an inflated configuration. Opposing portions of the first wall portion 706, and opposing portions of the second wall portion 708 can move away from one another as the wall portions 706, 708 assume a tensioned state, such as while the inflatable balloon 700 is in the inflated configuration. As fluid leaves the inflatable balloon 700, the first and/or second wall portions 706, 708 can return to a relaxed state as the first and/or second ring portions 702, 704 revert back to the deflated configuration. Opposing portions of the first wall portion 706, and opposing portions of the second wall portion 708 can contract and/or shrink, and/or move toward one another. In some instances, inflation and/or deflation of the first and second ring portions 702, 704 can act as an axial spring storing energy between the two portions of the first and/or the second wall portions 706, 708.

In some instances, the inflatable balloon 700 can be oriented in a target vessel, lumen, and/or chamber so as to reduce or prevent occlusion of the target vessel, lumen, and/or chamber. For example, the opening 710 extending through the ring-shaped inflatable balloon 700 can be oriented to be parallel or substantially parallel to a direction of blood flow. The opening 710 can be aligned with the direction of blood flow in the vessel, lumen, and/or chamber.

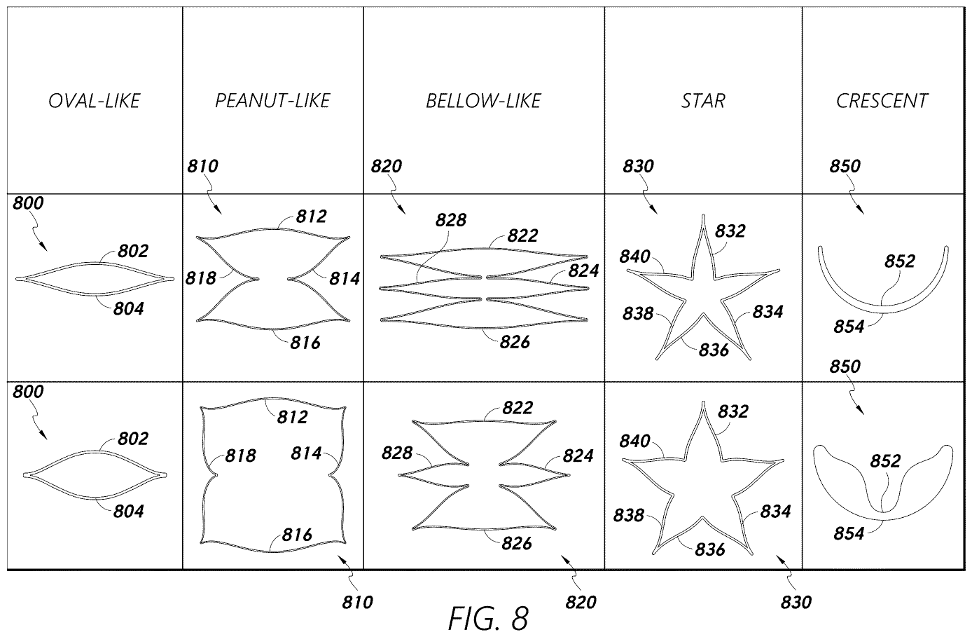

FIG. 8 is a chart showing cross sections of various examples of inflatable balloons 800, 810, 820, 830, 850. The cross sections can be taken along a plane perpendicular or substantially perpendicular to a longitudinal axis of the respective inflatable balloon. In some instances, a medical compliance device can comprise a first and/or second inflatable balloon having one or more of the cross sections described with reference to FIG. 8. The medical compliance device can comprise one or more other features of the one or more medical compliance devices described herein, such as medical compliance devices 100, 200, 300 described with reference to FIGS. 1, 2A, 2B, 2C, 3A and 3B. For example, a cross section of an inflatable balloon can comprise an oval shape, a peanut shape, a bellow shape, a star shape and/or a crescent shape. The upper row of cross sections in FIG. 8 shows an oval-shaped inflatable balloon 800, a peanut-shaped inflatable balloon 810, a bellow-shaped inflatable balloon 820, a star-shaped inflatable balloon 830, and a crescent-shaped inflatable balloon 850 in their respective deflated states. The lower row of cross sections shows the inflatable balloons 800, 810, 820, 830, 850 in their respective inflated states. In some instances, a relaxed state of the respective inflatable balloon can be a deflated configuration. For example, a first inflatable balloon of a medical compliance device can an inflatable balloon described with reference to FIG. 8. Alternatively, a relaxed state of the respective inflatable balloon can be an inflated configuration. For example, a second inflatable balloon of a medical compliance device can an inflatable balloon described with reference to FIG. 8.

Referring to FIG. 8, the oval-shaped inflatable balloon 800 can comprise a first and second wall portion 802, 804. The first and second wall portions 802, 804 can each have a convex curvature. In some instances, the first and second wall portions 802, 804 can be opposingly oriented. For example, the first and second wall portions 802, 804 can stretch and/or move away from one another to increase an internal volume and/or chamber defined at least in part by the first and second wall portions 802, 804 such that the oval-shaped inflatable balloon 800 can transform from a deflated configuration to an inflated configuration. The first and second wall portions 802, 804 can contract and/or move back toward one another such that the oval-shaped inflatable balloon 800 can revert back to the deflated configuration.

In some instances, the peanut-shaped inflatable balloon 810 can comprise a first, second, third and fourth wall portion 812, 814, 816, 818. In some instances, the first wall portion 812 and third wall portion 816 can each comprise at least a portion that are opposingly oriented. In some instances, the second wall portion 814 and the fourth wall portion 818 can each comprise at least a portion that are opposingly oriented. The second wall portion 814 and the fourth wall portion 818 can each comprise a fold configured to fold and unfold to allow the peanut-shaped inflatable balloon 810 to transform between deflated and inflated configurations. In some instances, the folds can form a concave exterior angle to allow folding inward of the respective wall portions 814, 818. The first and third wall portions 812, 816 can move toward and away from one another as the second and fourth wall portions 814, 818 fold and unfold, respectively.

Referring again to FIG. 8, the bellow-shaped inflatable balloon 820 can comprise a first, second, third and fourth wall portion 822, 824, 826, 828. In some instances, the first wall portion 822 and third wall portion 826 can each comprise at least a portion that are opposingly oriented. In some instances, the second wall portion 824 and the fourth wall portion 828 can each comprise at least a portion that are opposingly oriented. The second wall portion 824 and the fourth wall portion 828 can each comprise a plurality of folds configured to fold and unfold to allow the bellow-shaped inflatable balloon 820 to transform between deflated and inflated configurations. The plurality of folds can form alternating concave and convex exterior angles. The first and third wall portions 822, 826 can move toward and away from one another as the second and fourth wall portions 824, 828 fold and unfold, respectively.

In some instances, the star-shaped inflatable balloon 830 can comprise a first, second, third, fourth and fifth wall portion 832, 834, 836, 838, 840. For example, the star-shaped inflatable balloon 830 can be a five-cornered star shape. Ends of each of the first, second, third, fourth and fifth wall portions 832, 834, 836, 838, 840 can be coupled to respective ends of adjacent wall portions to form exterior convex angles of the star shape. For example, adjacent ones of the first, second, third, fourth and fifth wall portions 832, 834, 836, 838, 840 can be coupled to one another to form the corners and/or vertices of the star shape. Each of the first, second, third, fourth and fifth wall portions 832, 834, 836, 838, 840 can comprise a fold forming respective ones of the exterior concave angles of the star shape. Each fold can fold and unfold to allow the star-shaped inflatable balloon 830 to transform between deflated and inflated configurations.

In some instances, the crescent-shaped inflatable balloon 850 can comprise a first and second wall portion 852, 854. In some instances, while the crescent-shaped inflatable balloon 850 is in a deflated configuration, the first wall portion 852 can form a first curved portion of the crescent shape. The second wall portion 854 can form a second curved portion of the crescent shape. The second curved portion can have a larger radius of curvature than that of the first curved portion. For example, while the crescent-shaped inflatable balloon 850 is in the deflated configuration, the first wall portion 852 can have a radius of curvature smaller than that of the second wall portion 854. The first and/or second wall portion 852, 854 can stretch and/or move away from one another to transform the inflatable balloon 850 from the deflated to an inflated configuration. In some instances, one or more portions of the first wall portion 852 can stretch and/or move away from the second wall portion 854 to allow transformation of the crescent-shaped inflatable balloon 850 to assume the inflated configuration. FIG. 8 shows two portions of the first wall portion 852 stretching and/or displaced away from the second wall portion 854, such as forming two bulging and/or protruding portions, to provide the crescent-shaped inflatable balloon 850 in the inflated configuration. For example, while in the inflated configuration, the stretched portions of the first wall portion 852 can provide two expanded portions of the first internal volume and/or chamber, such as compared to adjacent portions of the first internal volume and/or chamber.

It will be understood that a medical compliance device can comprise an inflatable portion and/or member, including an inflatable balloon, comprising features of any one of the inflatable balloons described herein alone or in combination. For example, a medical compliance device can comprise a first inflatable balloon and second inflatable balloon each having features of one or more inflatable balloons described herein. In some instances, the first inflatable balloon can have features of an inflatable balloon described herein and the second inflatable balloon can have features of another inflatable balloon described herein. In some instances, the first and second inflatable balloons can have one or more features of an inflatable balloon described herein. For example, the first inflatable balloon can comprise features of the inflatable balloon while in a deflated configuration. The second inflatable balloon can comprise features of the inflatable balloon while in an inflated configuration.

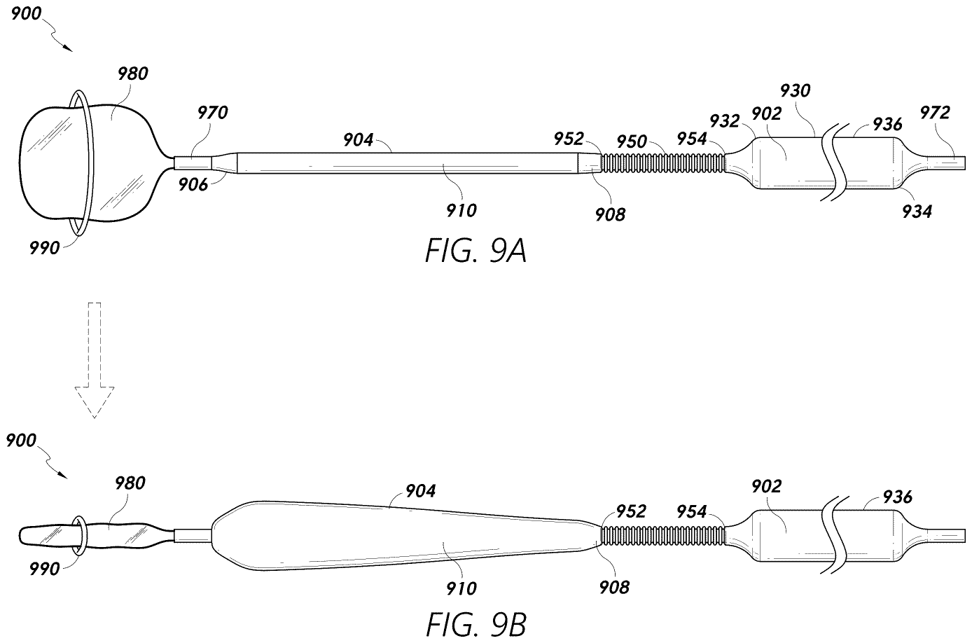

FIGS. 9A and 9B show an example of a medical implant system 900 comprising a fluid reservoir 980 and a medical compliance device 902. The fluid reservoir 980 can be coupled to the medical compliance device 902 to allow fluid delivery into the medical compliance device 902 at or proximate to the target site for the medical compliance device 902, including during and/or post implantation of the medical compliance device 902. In some instances, the fluid reservoir 980 allows adjustment in the fluid level within the medical compliance device 902. In some instances, the medical compliance device 902 can comprise one or more features of one or more medical compliance devices described herein. For example, the medical compliance device 902 can comprise one or more features of the medical compliance devices 100, 200, 300 described with reference to FIGS. 1, 2A, 2B, 2C, 2D, 3A and 3B. In some instances, the medical compliance device can comprise a first inflatable balloon 904 having a first internal volume and/chamber, and a second inflatable balloon 930 having a second internal volume and/chamber. A connector 950 comprising a lumen and/or conduit can extend between the first and second inflatable balloons 904, 930 to allow transfer of fluid between the first and second inflatable balloons 904, 930. A first end portion 952 of the connector 950 can be coupled to a second end portion 908 of the first inflatable balloon 904. A second end portion 954 of the connector 950 can be coupled to a first end portion 932 of the second inflatable balloon 930. In some instances, the first inflatable balloon 904 can be in a deflated configuration while in a relaxed state. The first inflatable balloon 904 can be in an inflated configuration while in a tensioned state. The second inflatable balloon 930 can be in an inflated configuration while in a relaxed state, and in a deflated configuration while in a tensioned state. FIGS. 9A and 9B show the fluid reservoir 980 coupled to an end portion of the first inflatable balloon 904. For example, the fluid reservoir 980 can be coupled to a first end tubular member 970 coupled to a first end portion 906 of the first inflatable balloon 904. The first end tubular member 970 can comprise a port through which fluid from the fluid reservoir 980 can be transferred. Alternatively or in combination, a fluid reservoir can be coupled to an end portion of the second inflatable balloon 930. For example, the fluid reservoir 980 can be coupled to a second end tubular member 972 coupled to a second end portion 934 of the second inflatable balloon 930. In some instances, the first end tubular member 970 can define a lumen through which fluid from the fluid reservoir 980 can be delivered into the first inflatable balloon 904. In some instances, a one-way valve within the lumen can facilitate control of fluid flow into the first inflatable balloon 904. Fluid from the fluid reservoir 980 can be deployed into the first inflatable balloon 904 during and/or after an implantation procedure. For example, fluid from the fluid reservoir 980 can facilitate remote future adjustments in fluid level of the medical compliance device 902.

FIG. 9A shows the fluid reservoir 980 in a full state. FIG. 9B shows the fluid reservoir 980 in an empty state. Delivery of fluid from the fluid reservoir 980 can comprise compressing and/or squeezing the fluid reservoir, such as using a sinching component 990 to circumferentially compress a portion of the fluid reservoir 980. FIG. 9A shows the sinching component 990 in a relaxed state and comprising at least a portion disposed around the fluid reservoir 980. FIG. 9B shows tensioning of the sinching component 990 such that the sinching component 990 compresses the fluid reservoir 980 to push fluid into the first inflatable balloon 904, inflating the first inflatable balloon 904. Various methods for expelling the fluid from the fluid reservoir 980 can be applicable, including for example a magnetic field remote activation, and or electromechanical system activation of screw-syringe piston-like mechanism. In some instances, the medical compliance device 902 can be empty prior to infusion of fluid from the fluid reservoir 980. Alternatively, the medical compliance device 902 can have an amount of fluid contained therein prior to addition of fluid from the fluid reservoir 980. Addition of fluid from the fluid reservoir 980 into the medical compliance device 902 can be used to provide an adjustment in the amount of fluid within the medical compliance device 902.

The medical compliance device 902 can comprise one or more other features of the medical compliance devices 200, 300 described with reference to FIGS. 2A, 2B, 2C, 2D, 3A and 3B. For example, wall portions 910, 936 can form at least a portion of the first and second inflatable balloons 904, 930. In some instances, the wall portions 910, 936 can at least partially define the first and second internal volumes and/or chambers, respectively. A lateral cross section of the first inflatable balloon 904 can comprise a rectangular or substantially rectangular shape while the first inflatable balloon 904 is in the deflated configuration. A lateral cross section of the second inflatable balloon 930 in the inflated configuration can comprise one or more convex edges. In some instances, the lateral cross section of the second inflatable balloon 930 can comprise an oval shape, including a circular or substantially circular shape. In some instances, the second inflatable balloon 930 in the inflated configuration can comprise a cylindrical shape. The lateral cross sections can be taken along a plane perpendicular or substantially perpendicular to a longitudinal axis of the medical compliance device 902 and/or of the respective inflatable balloon. In some instances, a cross section of the first inflatable balloon 904 along a plane containing the longitudinal axis of the first inflatable balloon 904 can comprise a rectangular or substantially rectangular shape. In some instances, the medical compliance device 902 can comprise a valve at least partially disposed within the connector 950. The valve can be configured to remain closed while the arterial pressure is below a threshold value and assume an open state when the arterial pressure is at and/or above the threshold, thereby reducing or preventing fluid flow from the second inflatable balloon 930 into the first inflatable balloon 904 during at least a portion a diastole phase.

### Additional Description of Examples

Provided below is a list of examples, each of which may include aspects of any of the other examples disclosed herein. Furthermore, aspects of any example described above may be implemented in any of the numbered examples provided below.

Example 1: A medical compliance device can comprise a first inflatable portion having a first wall portion defining at least a portion of the first inflatable portion, the first inflatable portion being configurable in a first deflated configuration in which the first wall portion is in a first relaxed state and a first inflated configuration in which the first wall portion is in a first tensioned state. The device can include a second inflatable portion comprising a second wall portion defining at least a portion of the second inflatable portion, the second inflatable portion being configurable in a second inflated configuration in which the second wall portion is in a second relaxed state and a second deflated configuration in which the second wall portion is in a second tensioned state. The device can include a connector extending between the first and second inflatable portions and defining a lumen configured to provide fluid communication between the first and second inflatable portions.

Example 2: The device of any example herein, in particular example 1, wherein the first inflatable portion comprises a first inflatable balloon and the second inflatable portion comprises a second inflatable balloon.

Example 3: The device of any example herein, in particular example 1 or 2, wherein a lateral cross section of the first wall portion in the first relaxed state comprises first and second opposing portions that are parallel to one another, the lateral cross section being taken along a plane perpendicular to a longitudinal axis of the first inflatable portion.

Example 4: The device of any example herein, in particular example 3, wherein the lateral cross section of the first wall portion in the first relaxed state comprises a rectangular shape.

Example 5: The device of any example herein, in particular examples 1 to 4, wherein a cross section of the first wall portion in the first relaxed state along a plane containing a longitudinal axis of the first inflatable portion comprises a rectangular shape.

Example 6: The device of any example herein, in particular examples 1 to 5, wherein a lateral cross section of the first wall portion in the first relaxed state comprises at least one of an oval shape, peanut shape, bellow shape, star shape and crescent shape, the lateral cross section being taken along a plane perpendicular to a longitudinal axis of the first inflatable portion.

Example 7: The device of any example herein, in particular examples 1 to 6, wherein a lateral cross section of the second wall portion in the second relaxed state comprises at least one convex edge, the lateral cross section being taken along a plane perpendicular to a longitudinal axis of the second inflatable portion.

Example 8: The device of any example herein, in particular example 7, wherein the lateral cross section of the second wall portion in the second relaxed state comprises a circle.

Example 9: The device of any example herein, in particular examples 1 to 8, wherein the first and second wall portions each comprise a shape-memory material.

Example 10: The device of any example herein, in particular example 9, wherein the shape-memory material comprises nitinol.

Example 11: The device of any example herein, in particular examples 1 to 10, wherein the connector comprises a tubular form.

Example 12: The device of any example herein, in particular examples 1 to 11, wherein a lateral dimension of the connector is smaller than a lateral dimension of at least one of the first and second inflatable portions.

Example 13: The device of any example herein, in particular examples 1 to 12, wherein a wall portion defining at least a portion of the connector comprises circumferential ridges oriented perpendicularly relative to a longitudinal axis of the connector.

Example 14: The device of any example herein, in particular examples 1 to 13, further comprising a valve having a portion configured to be disposed within the connector and assume a closed state in a relaxed state to occlude a fluid flow pathway between the first and second inflatable portions.

Example 15: The device of any example herein, in particular example 14, wherein the valve comprises a ball positioned over an end of a spring, the valve being configurable to be in the closed state with the spring being in a relaxed state and the ball being positioned along the fluid flow pathway to occlude the fluid flow pathway.

Example 16: The device of any example herein, in particular example 15, wherein the spring is configured to be compressed in a tensioned state to move the ball toward the first inflatable portion to a location along the fluid flow pathway where the ball does not occlude the fluid flow pathway and provide the valve in an open state.

Example 17: The device of any example herein, in particular examples 1 to 16, wherein the first inflatable portion comprises an inflatable balloon having a plurality of folds to allow the inflatable balloon to expand and contract along a linear dimension parallel to a longitudinal axis of the inflatable balloon.

Example 18: The device of any example herein, in particular examples 1 to 17, wherein the first inflatable portion comprises an inflatable balloon comprising a first and a second end portion, at least one of the first and second end portion being configured to rotate relative to the other around a longitudinal axis of the inflatable balloon to provide inflated and deflated configurations.

Example 19: The device of any example herein, in particular examples 1 to 18, wherein the first inflatable portion comprises a ring-shaped inflatable balloon, opposing portions of the ring-shaped balloon being configured to inflate or deflate.

Example 20: The device of any example herein, in particular examples 1 to 19, wherein the first inflatable portion is configured to be positioned in the inferior vena cava and the second chamber is configured to be positioned in the aorta.

Example 21: The device of any example herein, in particular examples 1 to 20, wherein the first inflatable portion is configured to be coupled to a fluid reservoir comprising fluid configured to be transferred into the first inflatable portion.

Example 22: A medical compliance device can comprise a first inflatable balloon configured to be disposed within a venous vessel. The first inflatable balloon can comprise a first internal chamber. The device can include a second inflatable balloon configured to be disposed within an arterial vessel and being configurable to assume an inflated configuration in a relaxed state and a deflated configuration in a tensioned state, the second inflatable balloon comprising a second internal chamber. The device can include a connector extending between the first and second inflatable balloons to provide fluid communication between the first and second internal chambers, and a fluid contained within the device. Compressive forces exerted upon of the second inflatable balloon by arterial pressure can be configured to provide the second inflatable balloon in the deflated configuration and push fluid from the second internal chamber into the first internal chamber, and the second inflatable balloon being configured to revert to the inflated configuration after removal of the compressive forces to allow the fluid to return to the second internal chamber.

Example 23: The device of any example herein, in particular example 22, wherein the device can be sealed and can comprise the fluid pre-loaded therein.

Example 24: The device of any example herein, in particular example 22 or 23, wherein at least one of the first and second inflatable balloons can be configured to be coupled to a fluid reservoir.

Example 25: The device of any example herein, in particular examples 22 to 24 further comprising a valve at least partially received within the connector, the valve being configured to assume an open state during at least a portion of systole to allow fluid flow from the second internal chamber to the first internal chamber, and a closed state during at least a portion of diastole.

Example 26: The device of any example herein, in particular example 25, wherein the valve can comprise a spring disposed along a fluid flow pathway within a lumen of the connector, a first end of the spring being oriented toward the first inflatable balloon and a second end oriented toward the second inflatable balloon. The valve can include a ball positioned against the second end of the spring, the spring being configured to shorten as arterial pressure increases to position the ball toward the first inflatable balloon at a location along the fluid flow pathway where the ball does not occlude the fluid flow pathway and provide the valve in an open state. The spring can be configured to lengthen as arterial pressure decreases to position the ball along the fluid flow pathway at a location to occlude the fluid flow pathway and provide the valve in a closed state.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, FIGURE, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method of modulating vascular flow in a patient, the method comprising:
providing a metallic compliance-enhancement device adapted to have a volume of fluid sealed therein, the metallic compliance-enhancement device comprising:
a first elongated inflatable nitinol balloon having a first inflated diameter;
a second elongated inflatable nitinol balloon having a second inflated diameter, each of the first balloon and the second balloon having a fluid-sealed terminus at an outer end and an open fluid port at an inner second end; and
a nitinol connector tube fluidly coupling the open fluid port of the first balloon to the open fluid port of the second balloon, the connector tube having a third diameter that is less than the first and second inflated diameters;
positioning the first elongated inflatable balloon within an arterial blood vessel of anatomy of a patient;
spanning a distance between the arterial blood vessel and a venous blood vessel of the anatomy with the connector tube;
positioning the second elongated inflatable balloon within the venous blood vessel;
exposing the first balloon to systolic arterial pressure within the arterial blood vessel, thereby causing the first balloon to compress radially inwardly and force fluid from within the first balloon to pass out of the open fluid port of the first balloon, through the connector tube, and into the second balloon through the open fluid port of the second balloon to cause the second balloon to expand radially outwardly within the venous blood vessel and increase blood flow therein; and
cyclically transferring fluid between the first and second balloons in response to systolic and diastolic pressure changes;
wherein each of the first and second elongated inflatable nitinol balloons is adapted to undergo a longitudinally-distributed, outward-expanding deformation that increases an outer dimensional profile of the balloon when fluid volume is increased therein.

2. The method of claim 1, wherein:
positioning the first balloon within the arterial blood vessel involves orienting the terminus of the first balloon in a first direction; and
positioning the second balloon within the venous blood vessel involves orienting the terminus of the second balloon in a second direction opposite the first direction.

3. The method of claim 1, wherein:
the first balloon has a shape-memory configuration that is biased to an expanded state; and
the second balloon has a shape-memory configuration that is biased to a compressed state.

4. The method of claim 1, wherein each of the first and second balloons is adapted to assume:
an expanded inflated configuration when a majority of the volume of fluid is passed into a balloon volume thereof; and
a compressed deflated configuration when the majority of the volume of fluid is discharged from the balloon volume.

5. The method of claim 4, wherein:
the expanded inflated configuration of the second balloon has an expanded star cross-sectional shape; and
the compressed deflated configuration of the second balloon has a compressed star cross-sectional shape.

6. The method of claim 4, wherein:
the expanded inflated configuration of the second balloon has an expanded bellows cross-sectional shape; and
the compressed deflated configuration of the second balloon has a compressed bellows cross-sectional shape.

7. The method of claim 4, wherein:
the expanded inflated configuration of the second balloon has an expanded crescent cross-sectional shape; and
the compressed deflated configuration of the second balloon has a compressed crescent cross-sectional shape.

8. The method of claim 1, wherein at least one of the first balloon or the second balloon, in a deflated compressed state, has an oval cross-sectional shape.

9. The method of claim 1, wherein:
the first balloon is tensioned when in a compressed deflated state; and
the second balloon is tensioned when in an expanded inflated state.

10. The method of claim 1, wherein the connector tube comprises a plurality of circumferential ridges associated with an outer diameter of the connector tube.

11. The method of claim 10, further comprising positioning the connector tube using frictional contact between one or more of the plurality of circumferential ridges and tissue of the anatomy.

12. The method of claim 1, wherein the connector tube includes a valve configured to block flow through connector tube when fluid pressure within at least a portion of the connector tube is below a threshold.

13. The method of claim 12, wherein the valve comprises:
a spring that is coaxial with the connector tube; and
a ball configured to compress the spring in response to fluid pressure forces.

14. The method of claim 1, further comprising, after positioning the second elongated inflatable nitinol balloon within the venous blood vessel, injecting the compliance-enhancement device with the volume of fluid through an injection port associated with the outer end of the first balloon or the second balloon.

15. A method of modulating vascular flow in a patient, the method comprising:
placing a first elongated inflatable balloon within an arterial blood vessel of anatomy of a subject, the first balloon having a sealed outer end and a fluidly-open inner end;
spanning a distance between the arterial blood vessel and a venous blood vessel of the anatomy with a connector tube coupled to the inner end of the first balloon at a first end of the connector tube and to a fluidly-open inner end of a second elongate inflatable balloon at a second end of the connector tube;
positioning the second balloon within the venous blood vessel;
exposing the first balloon to systolic arterial pressure within the arterial blood vessel that is greater than a fluid pressure within the first balloon, thereby causing:
the first balloon to compress radially inwardly to a radially-compressed deflated state, thereby forcing a portion of fluid from within the first balloon to pass through the connector tube and into the second balloon; and
fluid pressure within the second balloon to increase beyond pressure in the venous blood vessel such that the second balloon expands radially outwardly from a deflated state to a radially-expanded inflated state; and
transferring the portion of fluid back from the second balloon to the first balloon as venous pressure rises and radially compresses the second balloon to force the portion of fluid out of the second balloon and back into the first balloon.

16. The method of claim 15, wherein the connector tube is oriented perpendicularly to axes of the first balloon and the second balloon.

17. The method of claim 15, wherein the first balloon, the connector tube, and the second balloon together form a U-shaped implant as implanted in the anatomy.

18. A method of modulating vascular flow in a patient, the method comprising:
placing a first elongated inflatable balloon within an arterial blood vessel of anatomy of a subject, the first balloon being shape-memory-biased to a radially-expanded state and having a sealed outer end and a fluidly-open inner end;
spanning a distance between the arterial blood vessel and a venous blood vessel of the anatomy with a connector tube coupled to the inner end of the first balloon at a first end of the connector tube and to a fluidly-open inner end of a second elongate inflatable balloon at a second end of the connector tube;
positioning the second balloon within the venous blood vessel, the second balloon being shape-memory-biased to a radially-compressed state;
when the first balloon is in the radially-expanded inflated state, exposing the first balloon to systolic arterial pressure within the arterial blood vessel that overcomes a shape-memory bias force of the first balloon, thereby causing:
the first balloon to compress radially inwardly to a tensioned radially-compressed deflated state and force a volume of fluid from within the first balloon to pass through the connector tube and into the second balloon; and
fluid pressure within the second balloon to overcome a shape-memory bias force of the second balloon and expand radially outwardly from the radially-compressed state to a tensioned radially-expanded inflated state; and
transferring the volume of fluid back from the second balloon to the first balloon as diastolic venous pressure rises and combines with the shape-memory bias force of the second balloon to force the volume of fluid out of the second balloon and back to the first balloon.

19. The method of claim 18, wherein the first balloon, second balloon, and connector tube are formed of a unitary physical vapor deposited nitinol film.

20. The method of claim 18, wherein the volume of fluid comprises saline solution.
